(19) United States
(12) Patent Application Publication
(10) Pub. No.: US 2026/0078158 A1
(43) Pub. Date: Mar. 19, 2026

(54) COMPOSITIONS COMPRISING CXCL10-DERIVED PEPTIDES AND METHODS OF USE THEREOF

(71) Applicant: University of Virginia Patent Foundation, Charlottesville, VA (US)

(72) Inventors: Molly A. Hughes, Charlottesville, VA (US); Matthew Crawford, Charlottesville, VA (US)

(73) Assignee: University of Virginia Patent Foundation, Charlottesville, VA (US)

(21) Appl. No.: 19/108,419

(22) PCT Filed: Sep. 5, 2023
(86) PCT No.: PCT/US2023/073488

§ 371 Date: Mar. 3, 2025

Related U.S. Application Data
(60) Provisional application No. 63/403,586, filed on Sep. 2, 2022.

(51) Int. Cl. C07K 14/52; A01N 63/50; A01P 1/00; A61K 38/00; A61K 47/64; A61L 26/00; A61P 31/02

(52) U.S. Cl. CPC C07K 14/522; A01N 63/50; A01P 1/00; A61K 47/64; A61L 26/0066; A61P 31/02; A61K 38/00

(57) ABSTRACT

Provided are peptides, modified peptides, fragments thereof, conjugates thereof, and polymers thereof that have antibacterial activity, including against multidrug-resistant bacteria. In some embodiments, the peptides include amino acid sequences as set forth in any of SEQ ID NOs: 2-151, modified peptides, fragments, and conjugates thereof, and any combination thereof. The peptides, modified peptides, fragments, and conjugates can be polymer-functionalized, encapsulated in a particle, embedded in and/or on a solid support, impregnated in a dressing, and/or is formulated for use in a nebulizer, for topical administration, and/or for systemic administration. Also provided are medical devices having an antibacterial agent embedded in and/or associated with a support layer, and methods for inhibiting growth of and/or killing bacteria, for treating or preventing community and/or nosocomial infections, for treating bacterial infections present in wounds, for treating pulmonary infections, for treating or preventing systemic bacterial infections, and for combination therapies with conventional antibiotics.

### CROSS REFERENCE TO RELATED APPLICATION

The presently disclosed subject matter claims priority to and the benefit of U.S. Provisional Patent Application Ser. No. 63/403,586, filed Sep. 2, 2022, the disclosure of which is herein incorporated by reference in its entirety.

### GRANT STATEMENT

This invention was made with government support under Grant No. R01 A1150941 awarded by National Institutes of Health. The Government has certain rights in the invention.

### REFERENCE TO SEQUENCE LISTING XML

The Sequence Listing XML associated with the instant disclosure has been electronically submitted to the United States Patent and Trademark Office via the Patent Center as a 156,065 byte UTF-8-encoded XML file created on Sep. 5, 2023 and entitled “3062_188_PCT.xml”. The Sequence Listing submitted via Patent Center is hereby incorporated by reference in its entirety.

### TECHNICAL FIELD

The presently disclosed subject matter relates to compositions and methods useful for treating and preventing bacterial infections. In particular, the presently disclosed subject matter relates to peptide-based compositions with enhanced bactericidal activity that can be administered to subject to treat and/or prevent bacterial infections.

### BACKGROUND

Since 1996, there has been a dramatic and alarming increase in the isolation of multi-drug resistant (MDR) bacteria, such as Klebsiella pneumoniae and Escherichia coli, from patients with bloodstream infections, pneumonias, and intra-abdominal infections. Multi-drug resistance typically denotes bacteria resistant to three or more classes of antibiotics. The increase in MDR bacteria has now been recognized throughout the world. These MDR bacteria are resistant to not only the cephalosporins but also to the carbapenems (imipenem, meropenem, ertapenem, and doripenem), which have traditionally been the last line of antimicrobial defense against the cephalosporin-resistant organisms. So-called carbapenem-resistant Enterobacteriaceae (CRE) applies to Enterobacteriaceae such as Klebsiella spp., Escherichia coli, Enterobacter spp., Citrobacter spp., Salmonella spp., Shigella spp., etc., which are characterized as being resistant to >3 classes of antibiotics, including carbapenems. Carbapenemases are enzymes that inactivate carbapenems, and include at least the following: Klebsiella pneumoniae carbapenemase (KPC), which is the most commonly encountered mechanism in United States; Oxacillinase (OXA-48), which is most commonly encountered in isolates from Europe and the Middle East; and New Delhi Metallo R-lactamase (NDM), which is most commonly encountered in Southern Asia, particularly in Pakistan and India, but also in the Balkans and Middle East. Of particular concern is that global distributions are spreading and evolving due to mobility and travel of humans. Genes for carbapenemases are located on chromosomes or, more commonly, plasmids, which are mobile, highly transmissible genetic elements found in many bacterial species. These organisms frequently carry multiple mechanisms of resistance in addition to carbapenemase, rendering them multi-drug resistant.

Given that the carbapenems are not effective against CRE, there are now very few antimicrobial options available. In the absence of any new antibiotics to combat these pathogens, healthcare workers are being forced to use older antibiotics such as colistin, which was used in the 1970's and has significant and serious side effects. Moreover, colistin-resistant bacteria are now being identified, reducing the available armamentarium of antimicrobials to one or zero antibiotics.

Much of the focus in the last two decades has been on resistance in Gram-positive organisms (e.g., methicillin-resistant Staphylococcus aureus (MRSA) and vancomycin-resistant Enterococcus (VRE)), although Gram-negative bacteria account for a large proportion of nosocomial infections. For example, in New York City, Gram-negative organisms account for at least 8% of nosocomial infections, and at least half of those are MDR, carbapenem-resistant bacteria.

The list of antibiotics available to treat infections with any of these MDR CRE organisms is limited from very few to none. Approximately 60/of hospital patients in the United States received at least one dose of an antibacterial, of which perhaps about 50% might be inappropriate and could be at least partially responsible for the rise in antimicrobial resistance. Antimicrobial resistance accounts for about 8 million additional days of hospitalization and contributes to the majority of almost 100,000 annual hospital-acquired infection (HAI) related deaths. From 1998 to 2009, for example, the annual costs associated with infections caused by MDR bacterial pathogens rose from about $4 billion to about $21-34 billion. Concurrently, the total number of new antibacterial agents being developed has dropped from about 16 from 1983-1987 to just 1 between 2008 and 2012. Novel approaches are critically needed for identifying new therapies for treating such pathogens, especially since simply producing newer generation antibiotics of the same class is unlikely to provide much benefit since cross-resistance can rapidly develop.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to peptides, peptide conjugates, and polymers thereof that have antibacterial activity.

In some embodiments, the presently disclosed subject matter relates to peptides comprising, consisting essentially of, or consisting of the amino acid sequence RFVRCTCI (SEQ ID NO: 42), RWVRCTCI (SEQ ID NO. 45). RYVRCTCI (SEQ ID NO: 46), RTVRCRCI (SEQ ID NO: 48), RTVRCMCI (SEQ ID NO: 59), RYVRCRCI (SEQ ID NO: 70), RRVRCRCI (SEQ ID NO: 72), RWVRCWCI (SEQ ID NO: 73), modified peptides thereof, and fragments thereof, optionally wherein one or more of the amino acids of the peptides, further optionally all of the amino acids of the peptides, are D-amino acids. In some embodiments, the peptides have bactericidal and/or bacteriostatic activity against a bacterium selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp. and Enterobacter cloacae; sexually-transmitted bacteria such as but not limited to Neisseria gonorrhoeae; enteric pathogens such as but not limited to Salmonella enterica serovars such as but not limited to Salmonella enterica serovar Typhi and Shigella flexneri; and biothreat agents such as but not limited to Bacillus anthracis in both vegetative and spore forms. In some embodiments, the bacterium is an MDR strain of Enterococcus faecium, Staphylococcus aureus, Klebsiella pneumoniae, Acinetobacter baumannii, Pseudomonas aeruginosa, Salmonella enterica, optionally Salmonella enterica serovar Typhi, or Shigella flexneri. In some embodiments, the peptides comprise, consist essentially of, or consist of the amino acid sequence RTVRCRCI (SEQ ID NO: 48), RTVRCMCI (SEQ ID NO: 59), or RYVRCRCI (SEQ ID NO: 70), and/or are modified peptides thereof, and/or fragments thereof.

In some embodiments, the presently disclosed subject matter also relates to peptides comprising, consisting essentially of, or consisting of the amino acid sequence RTVRCRCI (SEQ ID NO: 48), RTVRCMCI (SEQ ID NO: 59), or RYVRCRCI (SEQ ID NO: 70), modified peptides thereof, and fragments thereof. In some embodiments, one or more, optionally all, amino acids are D-amino acids.

In some embodiments, a peptide of the presently disclosed subject matter is polymer-functionalized, encapsulated in a particle, embedded in and/or on a solid support, optionally wherein the peptide is formulated for release from the solid support, impregnated in a dressing, optionally wherein the peptide is formulated for release from the dressing, and/or is formulated for use in a nebulizer, for topical administration, and/or for systemic administration.

In some embodiments, a peptide of the presently disclosed subject matter is a modified peptide that comprises a modification at the N-terminus, the C-terminus, or both the N-terminus and the C-terminus. In some embodiments, wherein the N-terminal and/or the C-terminal modification is selected from the group consisting of an addition or an aminohexanoic acid (AHX), an azido alanine (Ala(N₃)), a benzoylbenzoic acid (4-BBA), an azido phenylalanine (Phe(4-N₃)), a propargylglycine (PRA), or diazirine-containing amino acid residues, and/or is a replacement of a phenylalanine with an azido phenylalanine (Phe(4-N₃)). In some embodiments, the modification at the N-terminus and/or the C-terminus comprises insertion of a chemically-modified and/or unnatural amino acid at or near (e.g., within 1, 2, 3, 4, 5, or 6 amino acids) of the N- and/or C-terminus of a peptide of the presently disclosed subject matter. In some embodiments, the peptide that is modified is selected from the group consisting of SEQ ID NOs: 19, 20, 40, 42, AND 58.

The presently disclosed subject matter also relates in some embodiments to conjugates comprising a first peptide comprising, consisting essentially of, or consisting of the amino acid sequence RTVRCRCI (SEQ ID NO: 48), RTVRCMCI (SEQ ID NO: 59), or RYVRCRCI (SEQ ID NO: 70), a modified peptide thereof, or a fragment thereof, conjugated to a second peptide comprising, consisting essentially of, or consisting of the amino acid sequence of KNLLKAVSKERSKRSP (SEQ ID NO: 8) or PESKAIKNLLKAVSKERSKRSPSEQ (SEQ ID NO: 9), a modified peptide thereof, or a fragment thereof. In some embodiments, the first peptide is directly conjugated to the second peptide or the first and second peptides are indirectly conjugated to each other via a linker. In some embodiments, the linker is a peptide linker comprising 1-9 amino acids, optionally wherein the 1-9 amino acids are each individually selected from the group consisting of glycine and serine. In some embodiments, the conjugate is polymer-functionalized, encapsulated in a particle, embedded in and/or on a solid support, optionally wherein the peptide is formulated for release from the solid support, impregnated in a dressing, optionally wherein the peptide is formulated for release from the dressing, and/or is formulated for use in a nebulizer, for topical administration, and/or for systemic administration.

In some embodiments, the presently disclosed subject matter also relates to conjugates comprising: one or more peptides comprising, consisting essentially of, or consisting of the amino acid sequence RTVRCRCI (SEQ ID NO: 48); and/or one or more peptides comprising, consisting essentially of, or consisting of the amino acid sequence RTVRCMCI (SEQ ID NO: 59); and/or one or more peptides comprising, consisting essentially of, or consisting of the amino acid sequence or RYVRCRCI (SEQ ID NO: 70); and/or modified peptides and/or fragments thereof; and/or combinations thereof, wherein each peptide present in the conjugate is covalently linked to at least one other peptide via a non-peptide linker, a peptide linker, or a cysteine-cysteine linkage. In some embodiments, the conjugate comprises a branched conjugate, a flanking conjugate, a single conjugate, a linear polymer, a bottlebrush polymer, or any combination thereof. In some embodiments, the conjugate is polymer-functionalized, encapsulated in a particle, embedded in and/or on a solid support, optionally wherein the peptide is formulated for release from the solid support, impregnated in a dressing, optionally wherein the peptide is formulated for release from the dressing, and/or is formulated for use in a nebulizer, for topical administration, and/or for systemic administration. In some embodiments, at least one peptide is a modified peptide that comprises a modification at the N-terminus, the C-terminus, or both the N-terminus and the C-terminus. In some embodiments, the N-terminal and/or the C-terminal modification is selected from the group consisting of an addition or an aminohexanoic acid (AHX), an azido alanine (Ala(N₃)), a benzoylbenzoic acid (4-BBA), an azido phenylalanine (Phe(4-N₃)), and a propargylglycine (PRA), or diazirine-containing amino acid residues, and/or is a replacement of a phenylalanine with an azido phenylalanine (Phe(4-N₃)). In some embodiments, the peptide that is modified is selected from the group consisting of SEQ ID NOs: 19, 20, 40, 42, and 58.

The presently disclosed subject matter also relates in some embodiments to pharmaceutical compositions comprising, consisting essentially of, or consisting of a peptide as disclosed herein, a conjugate as disclosed herein, or any combination thereof, and a pharmaceutically acceptable carrier, diluent, or excipient. In some embodiments, the pharmaceutical composition is pharmaceutically acceptable for use in a human.

The presently disclosed subject matter also relates in some embodiments to medical devices comprising a support layer with an antibacterial agent embedded therein or associated therewith, wherein the antibacterial agent comprises a peptide and/or a conjugate of the presently disclosed subject matter. In some embodiments, the medical device is a wound dressing. In some embodiments, the peptide, chimeric peptide, and/or conjugate is encapsulated in a particle that is embedded in or associated with the support layer.

The presently disclosed subject matter also relates in some embodiments to methods for inhibiting the growth of and/or killing a bacterium. In some embodiments, the methods comprise contacting the bacterium with an effective amount of an antibacterial agent selected from the group consisting of a peptide and/or a conjugate of the presently disclosed subject matter. In some embodiments, the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to a Salmonella enterica serovars such as but not limited to Salmonella enterica serovar Typhi and Shigella flexneri; and biothreat agents such as but not limited to Bacillus anthracis in both vegetative and spore forms.

In some embodiments, the presently disclosed subject matter also relates to methods for treating a bacterial infection present in a wound. In some embodiments, the methods comprise contacting the wound with an effective amount of a composition comprising one or more peptides, each peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof.

In some embodiments, the presently disclosed subject matter also relates to methods for treating a pulmonary infection in a subject. In some embodiments, the methods comprise administering to a subject in need thereof an effective amount of a composition comprising one or more peptides, each peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof. In some embodiments, the composition is administered to the subject intranasally, by inhalation, optionally wherein the one or more peptides in the composition is/are aerosolized, or a combination thereof.

In some embodiments, the presently disclosed subject matter also relates to methods for treating or preventing a systemic bacterial infection in a subject. In some embodiments, the methods comprise administering to a subject in need thereof an effective amount of a composition comprising one or more peptides, each peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof.

In some embodiments, the presently disclosed methods further comprise administering to the subject a conventional antibiotic.

In some embodiments, the presently disclosed subject matter also relates to methods for inhibiting the growth of a biofilm. In some embodiments, the methods comprise contacting the biofilm with an effective amount of an antibacterial agent selected from the group consisting of a peptide and/or a conjugate as disclosed herein, or any combination thereof.

In some embodiments, the presently disclosed subject matter relates to uses of the presently disclosed peptides and/or conjugates, or any combination thereof, for preventing or treating a bacterial infection.

In some embodiments, the presently disclosed subject matter relates to compositions comprising, consisting essentially of, or consisting of a peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof, a conjugate thereof, a polymer thereof, or a combination thereof.

Accordingly, in some embodiments the presently disclosed subject matter relate to uses of the peptides, modified peptides, fragments thereof, conjugates thereof, and/or polymers thereof, and/or any combination thereof, for preventing or treating a bacterial infection.

Accordingly, it is an object of the presently disclosed subject matter to provide methods and compositions for treating and/or preventing infection by bacteria, in some embodiments MDR bacteria.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Detailed Description and Figures.

### BRIEF DESCRIPTION OF THE FIGURES

FIG. 1 is a structural representation of the human CXCL10 polypeptide. The CXCL10 polypeptide (top section of FIG. 1) consists of an unstructured N-terminal region responsible for interaction with the cellular receptor CXCR3, three antiparallel β-sheets, and an amphipathic C-terminal α-helix. The bottom section of FIG. 1 is a depiction of the production of an initial hCXCL10-derived peptide library. Synthetic 14- to 22-mer overlapping peptides were generated from the primary amino acid sequence of mature human CXCL10 (SEQ ID NO: 1). The amino acid sequences of Peptides P1-P6, P8, and P9 (SEQ ID NOs: 2-9, respectively) are shown. No Peptide P7 was synthesized.

FIG. 2 is a bar graph showing the bactericidal effects of Peptide P1 (SEQ ID NO: 2; black boxes) against the listed bacteria by percent survival reflected in relative fluorescence units (RFU) as compared to untreated controls. Peptide P5 (SEQ ID NO: 6) is included as a peptide-treated negative control. 50 μM peptide in RPMI medium; n=3-4 per experiment. ns: not significant. nd: none detected. ***: p<0.001.

FIG. 3 is a series of graphs assessing the host-targeted effects of Peptide P1 (SEQ ID NO: 2) including red blood cell (RBC) hemolysis and human T-cell cytotoxicity (50 μM peptide in RPMI medium; n=3), as well as chemoattraction of CXCR3-expressing primary human T-cells at the indicated peptide dilutions (n=3). Peptide P5 (SEQ ID NO: 6) is included as a peptide-treated negative control. ns: not significant. ***: p<0.001.

FIG. 4 is a bar graph of percent survival of K. pneumoniae in the presence of 50 μM of N- and C-terminal deletions of Peptide P1 (SEQ ID NO: 2) in RPMI medium (n=3-4). Peptide L8 (SEQ ID NO: 19) is identified as the minimal active sequence, and Peptide P5 (SEQ ID NO: 6) is included as a peptide-treated negative control. Other peptides include PLSRTVRCTCISI (SEQ ID NO: 10), LSRTVRCTCISI (SEQ ID NO: 11), SRTVRCTCISI (SEQ ID NO: 12), RTVRCTCISI (SEQ ID NO: 13), TVRCTCISI (SEQ ID NO: 14), VRCTCISI (SEQ ID NO: 15), RCTCISI (SEQ ID NO: 16), CTCISI (SEQ ID NO: 17), RTVRCTCIS (SEQ ID NO: 18), RTVRCTCI (SEQ ID NO: 19; Peptide L8), RTVRCTC (SEQ ID NO: 20), and RTVRCT (SEQ ID NO: 21). **: p<0.01. ***: p<0.001.

FIG. 5 is a bar graph and HPLC traces comparing the protease (trypsin and proteinase K) resistance of a peptide of SEQ ID NO: 19 with all L-amino acids (L8) to a peptide of SEQ ID NO: 19 with all D-amino acids (D8). The functional consequences of trypsin pretreatment were tested against K. pneumoniae treated with 50 μM peptide in RPMI medium (n=3-4). The effects of proteinase K on physical stability were tested in phosphate-buffered saline (n=2). ***: p<0.001.

FIG. 6 is a series of bar graphs comparing the antibacterial activities of a peptide of SEQ ID NO: 19 with all L-amino acids (L8) to a peptide of SEQ ID NO: 19 with all D-amino acids (D8). and Peptide P5 (SEQ ID NO: 6) is included as a peptide-treated negative control (top panel). In the bottom panel, the bactericidal activity of peptide D8 against K. pneumoniae or S. aureus is compared to that of a sequence-scrambled variant. In all cases, bacteria were treated with 50 μM peptide in RPMI medium (n=3-4). ***: p<0.001.

FIGS. 7A-7D show the results of testing Peptide D8 (SEQ ID NO: 19 with all D-amino acids) as a treatment of wound infection. K. pneumoniae infected wounds were treated topically with 10 μl of 1.2% Peptide D8 (SEQ ID NO: 19 with all D-amino acids) beginning 4 hours after infection and then 2× per day for 4 days; n=8 animals per group. FIG. 7A is a graph comparing percent survival at 0-21 days post-infection. The administration of Peptide D8 (SEQ ID NO: 19 with all D-amino acids; black squares) is compared to saline-alone (white squares). FIG. 7B is a photograph of an exemplary wound that was produced in a mouse to conduct the infection model. FIG. 7C is a series of photographs showing the resolution of infection and healing, or not, of exemplary wounds at 2, 4, 8, 14, and 21 days post-infection. FIG. 7D is a photograph of the growth of bacteria recovered from the wounds of animals treated with Peptide D8 (SEQ ID NO: 19 with all D-amino acids) or saline alone. ***: p<0.001.

FIG. 8 is a bar graph of percent survival of K. pneumoniae treated with 50 μM of Peptide D8 (SEQ ID NO: 19 with all D-amino acids) or individual alanine-scanning derivatives (SEQ ID NOs: 22-29 from top of Figure to the bottom) in RPMI medium (n=3-4). ns: not significant. **: p<0.01. ***: p<0.001.

FIGS. 9A-9C show the results of a Peptide D8 (SEQ ID NO: 19 with all D-amino acids) threonine-2 replacement scan. FIG. 9A is a bar graph of derivatives of Peptide D8 (SEQ ID NO: 19 with all D-amino acids) with each amino acid substitution at position 2. K. pneumoniae was treated with 50 μM peptide (all D-amino acids) in RPMI medium (n=3-4). FIGS. 9B and 9C are photographs of microplates inoculated with K. pneumoniae in Mueller-Hinton II broth for minimum inhibitory concentration (MIC) determination over the indicated peptide concentrations (n=3-4). Underlined substitution variants outperformed the parent Peptide D8. In FIG. 9A, the sequences are SEQ ID NOs: 19, 23, and 30-47 from top to bottom, respectively. In FIG. 9B, the sequences are SEQ ID NOs: 19, 23, 30, 31, 34, and 36-38 from top to bottom, respectively. In FIG. 9B, the sequences are SEQ ID NOs: 39-42, and 45-47 from top to bottom, respectively. *: p<0.05. ***: p<0.001.

FIGS. 10A-10C show the results of a Peptide D8 threonine-6 replacement scan. FIG. 10A is a bar graph of derivatives of Peptide D8 with each amino acid substitution at position 6. K. pneumoniae was treated with 50 μM peptide (all D-amino acids) in RPMI medium (n=3-4). FIGS. 10B and 10C are photographs of microplates inoculated with K. pneumoniae in Mueller-Hinton II broth for minimum inhibitory concentration (MIC) determination over the indicated peptide concentrations (n=3-4). Underlined substitution variants outperformed the parent Peptide D8. In FIG. 10A, the sequences are SEQ ID NOs: 19, 27, 48, 49, -65 from top to bottom, respectively. In FIG. 10B, the sequences are SEQ ID NOs: 19, 27, 48, 51, 53, and 55-57 from top to bottom, respectively. In FIG. 10C, the sequences are SEQ ID NOs: 58-60, and 62-65 from top to bottom, respectively. ***: p<0.001.

FIGS. 11A and 11B show the results of a Peptide D8 (SEQ ID NO: 19 with all D-amino acids) position 2/6 combinatorial replacements. Underlined substitution variants outperformed the parent Peptide D8 (SEQ ID NO: 19 with all D-amino acids). Peptides include T2F+T6R (SEQ ID NO: 66), T2F+T6M (SEQ ID NO: 67), T2W+T6R (SEQ ID NO: 68), T2W+T6M (SEQ ID NO: 69), T2Y+T6R (SEQ ID NO: 70), T2Y+T6M (SEQ ID NO: 71), T2R+T6R (SEQ ID NO: 72), and T2W+T6W (SEQ ID NO: 73).

FIG. 11C shows the disruption of bacterial killing that results when the two cysteine residues of Peptide D8 (SEQ ID NO: 19 with all D-amino acids) are replaced with cysteine analogs in which the thiol group is ‘capped’ with acetamidomethyl (ACM). Photographs of microplates inoculated with K. pneumoniae in Mueller-Hinton II broth for minimum inhibitory concentration (MIC) determination over the indicated peptide concentrations (n=3-4).

FIG. 12 is a summary of particular exemplary embodiments of the presently disclosed subject matter, including Peptides D8, P142, P145, P146, P171, P182, P194, P195 (SEQ ID NOs: 19, 42, 45, 46, 48, 59, 70, 72, and 73, respectively). ^{ad}: all D-amino acids.

FIG. 13 is a bar graph of percent RBC hemolysis of 100 μM Peptides D8, P142, P145, P146, P171, P182, P194, and P195 (SEQ ID NOs: 19, 42, 45, 46, 48, 59, 70, 72, and 73, respectively) in acetate (Ac) or formate (Fm) formulations in RPMI medium (n=3). The hemolytic peptide melittin was used as the positive control, and sterile water as the vehicle-alone control. ***: p<0.001. ^{ad}: all D-amino acids.

FIGS. 14A and 14B are a series of photographs showing the results of minimum inhibitory concentration (MIC) determinations over the indicated peptide concentrations for K. pneumoniae in Mueller-Hinton II broth (n=3-4). In FIG. 14A, the activity of lead-series peptide variants, produced as acetate or formate formulations, are compared. FIG. 14B summarizes lead optimization using acetate formulations, from discovery of the original peptide (P1), through determining the minimal active sequence (P60), conversion to D-amino acids (P73), and now the generation of substitution variants (P194). ^{ad}: all D-amino acids. In FIG. 14A, peptides include D8, P142, P145, P146, P171, P182, P194, and P195 (SEQ ID NOs: 19, 42, 45, 46, 48, 59, 70, 72, and 73, respectively). In FIG. 14B, peptides include P1 (SEQ ID NO: 2), P60 (SEQ ID NO: 19 with all L-amino acids), P73 (SEQ ID NO: 19 with all D-amino acids), and P194 (SEQ ID NO: 70 with all D-amino acids).

FIG. 15 shows the bactericidal activity of peptide variants (50 PM) containing chemically modified unnatural amino acid residues (e.g. photo-affinity and bioconjugation labels) against MDR K. pneumoniae or methicillin-resistant S. aureus in RPMI medium. (n=4). Peptides employed include P5 (SEQ ID NO: 6), P60 (SEQ ID NO: 19 with all L-amino acids), P73 (SEQ ID NO: 19 with all D-amino acids), P154 (SEQ ID NO: 19 with all D-amino acids and an N-terminal Biotin-Ahx moiety), P211 (SEQ ID NO: 40), P212 (SEQ ID NO: 58), P213 (SEQ ID NO: 74), P218 (SEQ ID NO: 19 with a C-terminal L-PRA moiety), P219 (SEQ ID NO: 19 with a C-terminal D-PRA moiety), P220 (SEQ ID NO: 20 with a C-terminal L-PRA moiety), P221 (SEQ ID NO: 20 with a C-terminal D-PRA moiety), P214 (SEQ ID NO: 42 with a L-Phe(4-N₃) derivative at amino acid 2), P215 (SEQ ID NO: 42 with a D-Phe(4-N₃) derivative at amino acid 2), P216 (SEQ ID NO: 76 with a L-Phe(4-N₃) derivative at amino acid 1), and P217 (SEQ ID NO: 76 with a D-Phe(4-N₃) derivative at amino acid 1).

FIGS. 16A and 16B show the bactericidal activity of peptide variants containing multiple chemically modified unnatural amino acid residues against MDR K. pneumoniae in RPMI medium. In FIG. 16A, bacterial survival following peptide (50 μM) exposure is reflected in relative fluorescence units (RFU) as compared to untreated controls in RPMI medium. (n=4). In FIG. 16B, photographs show the results of minimum inhibitory concentration (MIC) determinations over the indicated peptide concentrations in Mueller-Hinton II broth and comparing acetate and trifluoroacetic acid formulations (n=3-4). In FIG. 16A, peptides include P5 (SEQ ID NO: 6), P73 (SEQ ID NO: 19 with all D-amino acids), P227 (SEQ ID NO: 76 with a D-Phe(4-N₃) derivative at amino acid 1 and a D-PRA moiety at the C-terminus), P228 (SEQ ID NO: 42 with a D-Phe(4-N₃) derivative at amino acid 2 and a D-PRA moiety at the C-terminus), P229 (SEQ ID NO: 19 with a 4-Bba moiety at the N-terminus and a D-PRA moiety at the C-terminus), P230 (SEQ ID NO: 40 with a 4-Bba moiety at amino acid 2 and a D-PRA moiety at the C-terminus), P242 (SEQ ID NO: 77 with a D-Phe(4-N₃) derivative at the N-terminus and a D-PRA moiety at the C-terminus), and P245 (SEQ ID NO: 79 with a D-Phe(4-N₃) derivative at the N-terminus and a D-Ala(N₃) moiety at the C-terminus). In FIG. 16B, peptides include P244-TFA (TFA salt of SEQ ID NO: 78), P244-Ac (acetate salt of SEQ ID NO: 78), P250-TFA (TFA salt of SEQ ID NO: 83 with a photolabeled leucine at amino acid 2 and a D-Ala(N₃) moiety at the C-terminus), and P250-Ac (acetate salt of SEQ ID NO: 83 with a photolabeled leucine at amino acid 2 and a D-Ala(N₃) moiety at the C-terminus)

### BRIEF DESCRIPTION OF THE SEQUENCE LISTING

SEQ ID NO: 1 is the amino acid sequence of the mature form of a human CXCL10 (hCXCL10) polypeptide and corresponds to amino acids 22-98 of Accession No. NP_001556.2 of the GENBANK® biosequence database.

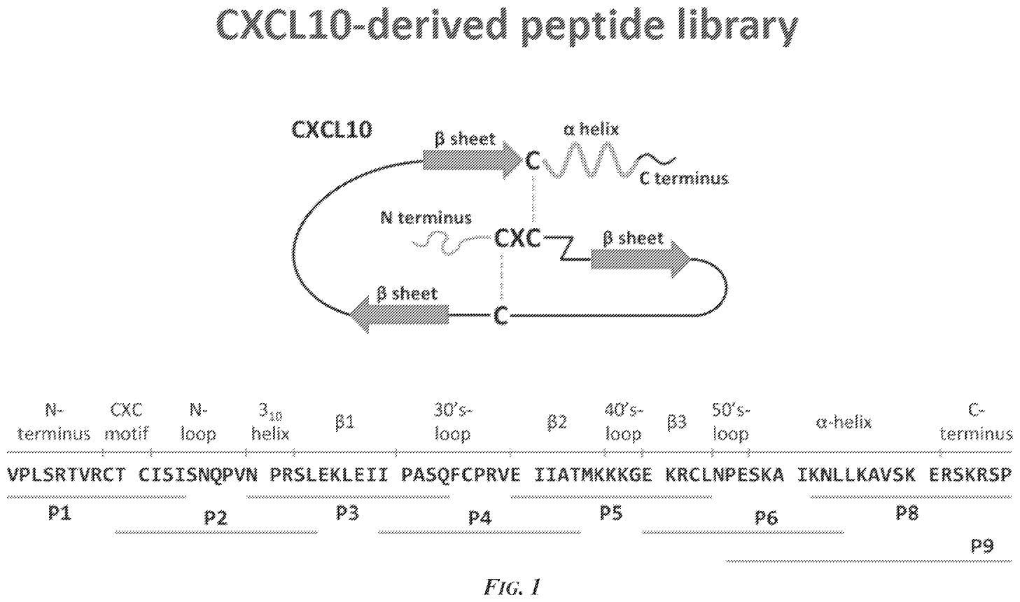

SEQ ID NOs: 2-9 are the amino acid sequences of overlapping peptides from SEQ ID NO: 1 as shown in FIG. 1. Particularly, SEQ ID NO: 2 is the amino acid sequence derived from the N-terminus of SEQ ID NO: 1 that has been shown to have antibacterial activity and immunomodulatory activity. This peptide is referred to herein as Peptide P1. It corresponds to amino acids 1-14 of SEQ ID NO: 1. SEQ ID NOs: 3-9 are the amino acid sequences of certain overlapping peptides derived from SEQ ID NO: 1 that together with SEQ ID NO: 2 span the entire sequence of SEQ ID NO: 1 as shown in FIG. 1. SEQ ID NOs: 3-9 are referred to herein as Peptides P2-P6, P8, and P9, respectively.

SEQ ID NOs: 10-17 are the amino acid sequences of peptides with N-terminal deletions of Peptide P1 (SEQ ID NO: 2). SEQ ID NO: 10 has the N-terminal amino acid of Peptide P1 (SEQ ID NO: 2) deleted, SEQ ID NO: 11 has the N-terminal two amino acids of Peptide P1 (SEQ ID NO: 2) deleted, SEQ ID NO: 12 has the N-terminal three amino acids of Peptide P1 (SEQ ID NO: 2) deleted, etc.

SEQ ID NO: 18 is the amino acid sequence of a peptide that corresponds to amino acids amino acids 5-13 of SEQ ID NO: 1 or SEQ ID NO: 2.

SEQ ID NO: 19 is the amino acid sequence of a peptide referred to herein as L8 or D8 that corresponds to amino acids 5-12 of SEQ ID NO: 1 or SEQ ID NO: 2.

SEQ ID NOs: 20 and 21 are the amino acid sequences of peptides with C-terminal deletions of SEQ ID NO: 19. SEQ ID NO: 20 has the C-terminal amino acid of SEQ ID NO: 19 deleted, and SEQ ID NO: 21 has the C-terminal two amino acids of SEQ ID NO: 219 deleted.

SEQ ID NOs: 22-29 are the amino acid sequences of alanine scanned variants of Peptide L8/D8 (SEQ ID NO: 19). In each of SEQ ID NOs: 22-29, one amino acid starting at the N-terminus was replaced with an alanine. For example, in SEQ ID NO: 22, the amino acid in position 1 of Peptide L8/D8 (SEQ ID NO: 19) was replaced with an alanine, in SEQ ID NO: 23, the amino acid in position 2 of Peptide L8/D8 (SEQ ID NO: 19) was replaced with an alanine, in SEQ ID NO: 24, the amino acid in position 3 of Peptide L8/D8 (SEQ ID NO: 19) was replaced with an alanine, etc.

SEQ ID NOs: 30-47 are the amino acid sequences of peptides with position 2 of Peptide L8/D8 (SEQ ID NO: 19) replaced with an amino acid other than alanine or threonine (SEQ ID NO: 19 has a threonine at position number 2 and SEQ ID NO: 23 has an alanine at position number 2) such that the remaining possible 18 amino acid changes are reflected in SEQ ID NOs: 30-47. A peptide with the amino acid sequence disclosed as SEQ ID NO: 42 is also referred to herein as Peptide P142 or just P142, a peptide with the amino acid sequence disclosed as SEQ ID NO: 45 is also referred to herein as Peptide P145 or just P145, a peptide with the amino acid sequence disclosed as SEQ ID NO: 46 is also referred to herein as Peptide P146 or just P146, a peptide with the amino acid sequence disclosed as SEQ ID NO: 48 is also referred to herein as Peptide P171 or just P171, and a peptide with the amino acid sequence disclosed as SEQ ID NO: 59 is also referred to herein as Peptide P182 or just P182.

SEQ ID NOs: 48-65 are the amino acid sequences of peptides with position 6 of Peptide L8/D8 (SEQ ID NO: 19) replaced with an amino acid other than alanine or threonine (SEQ ID NO: 19 has a threonine at position number 6 and SEQ ID NO: 27 has an alanine at position number 6) such that the remaining possible 18 amino acid changes are reflected in SEQ ID NOs: 48-65.

SEQ ID NOs: 66-73 are the amino acid sequences of peptides with double amino acid substitution variants of Peptide L8/D8 (SEQ ID NO: 19), with exemplary amino acid substitutions at both of positions 2 and 6 of SEQ ID NO: 19. The amino acids in SEQ ID NOs: 66-73 relative to SEQ ID NO: 19 are F2R6 (i.e., a phenylalanine at amino acid 2 and an arginine at amino acid 6), F2M6 (i.e., a phenylalanine at amino acid 2 and a methionine at amino acid 6), W2R6, W2M6, Y2R6 (peptide also referred to herein as P194), Y2M6, R2R6 (peptide also referred to herein as P195), and W2W6.

SEQ ID NOs: 74-90 are the amino acid sequences of exemplary modified peptides of the presently disclosed subject matter, including exemplary modified peptides that include one or more biotin moieties, Phe(4-N₃) moieties, PRA moieties, Ala(N₃) moieties, and/or diazirine Met/Leu/Lys moieties attached thereto, including certain exemplary modified peptides that include at least two of these moieties attached thereto, as set forth in Table 3

### DETAILED DESCRIPTION

The presently disclosed subject matter now will be described more fully hereinafter, in which some, but not all embodiments of the presently disclosed subject matter are described.

Indeed, the presently disclosed subject matter can be embodied in many different forms and should not be construed as limited to the embodiments set forth herein; rather, these embodiments are provided so that this disclosure will satisfy applicable legal requirements.

### I. General Considerations

Chemokines are chemotactic cytokines that are important regulators of leukocyte-mediated inflammation and immunity in response to a variety of diseases and infectious processes in the host. Chemokines are a superfamily of homologous 8-10 kDa heparin-binding proteins, originally identified for their role in mediating leukocyte recruitment. The four major families of chemokine ligands are classified on the basis of a conserved amino acid sequence at their amino terminus, and are designated CXC, CC, C, and CX3C sub-families (where “X” is a non-conserved amino acid residue).

The CXC chemokines are one of the largest families of chemokines, and each member of this group contains four cysteine residues. Most chemokines are small proteins (8-10 kDa in size), have a net positive charge at neutral pH, and share considerable amino acid sequence homology. Structurally, the defining feature of the CXC chemokine family is a motif of four conserved cysteine residues, the first two of which are separated by a non-conserved amino acid, thus constituting the Cys-X-Cys or ‘CXC’ motif. This family is further subdivided on the basis of the presence or absence of another three amino acid sequence, glutamic acid-leucine-arginine (the ‘ELR’ motif), immediately proximal to the CXC sequence. The ELR-positive (ELR⁺) CXC chemokines, which include IL-8/CXCL8, are potent neutrophil chemoattractants and promote angiogenesis. Among the ELR-negative (ELR⁻) CXC chemokines, CXCL9, CXCL10, and CXCL11 are potently induced by both type 1 and type 2 interferons (IFN-α/β and IFN-γ, respectively). These Interferon-inducible (ELR⁻) CXC chemokines are generated by a variety of cell types including monocytes, macrophages, lymphocytes, and epithelial cells, and are extremely potent chemoattractants for recruiting mononuclear leukocytes, including activated Th1 CD4 T cells, natural killer (NK) cells, NKT cells, and dendritic cells to sites of inflammation and inhibiting angiogenesis.

The chemokine receptors are a family of related receptors that are expressed on the surface of leukocytes as well as other cells. The shared receptor for CXCL9, CXCL10, and CXCL11 is CXCR3. Through their interaction with CXCR3, the ligands CXCL9, CXCL10, and CXCL11 are the major recruiters of specific leukocytes, including CD4 T cells, NK cells, and myeloid dendritic cells. Importantly, this chemokine ligand-receptor system is at the core of a positive feedback loop escalating Th1 immunity, whereby cytokines such as interleukin (IL)-12 and IL-18 (released by myeloid accessory cells) activate local NK cells to produce IFN-γ, thereby inducing the generation of CXCL9, CXCL10, and CXCL11, which then recruits CXCR3-expressing cells that act as a further source of IFN-γ, which then induces further production of CXCL9, CXCL10, and CXCL1l. Consistent with the importance of these interferon-inducible (ELR⁻) CXC chemokines in promoting Th1-mediated immunity, CXCR3 and its ligands have been documented to play a critical role in host defense against many microorganisms, including viruses, Mycobacterium tuberculosis, bacteria, and protozoa.

Independent of their role in CXCR3-dependent leukocyte recruitment, CXCL9, CXCL10, and CXCL11 have also been found to display direct antimicrobial properties that resemble those of defensins. These antimicrobial effects were first demonstrated in 2001 against Escherichia coli and Listeria monocytogenes. Subsequently, an increasing number of chemokines have been shown to have antimicrobial activity against various strains of bacteria and fungi, including Escherichia coli, Staphylococcus aureus, Candida albicans, and Cryptococcus neoformans.

The presently disclosed subject matter leverages the ELR-negative (ELR⁻) CXC chemokine/chemokine receptor system to provide compositions and methods for treating and/or preventing bacterial infections.

### II. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

A disease or disorder is “alleviated” if the severity of a symptom of the disease, condition, or disorder, or the frequency with which such a symptom is experienced by a subject, or both, are reduced by any measurable criterion. In some embodiments, a disease or disorder is “alleviated” if the severity of a symptom of the disease, condition, or disorder, or the frequency with which such a symptom is experienced by a subject, or both, are reduced to a condition that would be considered to be normal (i.e., absent).

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and includes humans. In the context of the invention, the term “subject” generally refers to an individual who will receive or who has received treatment for a condition characterized by the presence of bacteria (e.g., Bacillus anthracis in any stage of its growth cycle), or in anticipation of possible exposure to bacteria. As used herein, the terms “subject” and “patient” are used interchangeably, unless otherwise noted.

As used herein, the terms “neutralize” and “neutralization” when used in reference to bacterial cells or spores (e.g., B. anthracis cells and spores) refers to a reduction in the ability of the spores to germinate and/or cells to proliferate.

As used herein the term “bacterial spore” or “spore” is used to refer to any dormant, non-reproductive, but viable structure produced by some bacteria (e.g., Bacillus and Clostridium) in response to adverse environmental conditions.

As used herein, the term “treating a surface” refers to the act of exposing a surface to one or more compositions of the present invention. Methods of treating a surface include, but are not limited to, spraying, misting, submerging, wiping, and coating. Surfaces include organic surfaces (e.g., food products, surfaces of animals, skin, etc.) and inorganic surfaces (e.g., medical devices, countertops, instruments, articles of commerce, clothing, etc.).

As used herein, the terms “effective amount” and “therapeutically effective amount” are used interchangeably and refer to the amount that provides a therapeutic effect, e.g., an amount of a composition that is effective to treat or prevent pathological conditions, including signs and/or symptoms of disease, associated with a pathogenic organism infection (e.g., spore germination, bacterial growth, toxin production, etc.) in a subject.

The terms “bacteria” and “bacterium” refer to all prokaryotic organisms, including those within all of the phyla in the Kingdom Procaryotae. As used herein, the term “microorganism” refers to any species or type of microorganism, including but not limited to, bacteria, archaea, fungi, protozoans, mycoplasma, and parasitic organisms.

As used herein the term “colonization” refers to the presence of bacteria in a subject that are either not found in healthy subjects, or the presence of an abnormal quantity and/or location of bacteria in a subject relative to a healthy patient.

The term “stationary growth phase” as used herein defines the growth characteristics of a given population of microorganisms. During a stationary growth phase, the population of bacteria remains stable with the rate of bacterial division being approximately equal to the rate of bacterial death. This can be due to increased generation time of the bacteria. Accordingly, “stationary phase bacteria” are bacteria that are in a stationary growth phase. “Exponential phase bacteria” are bacteria that are rapidly proliferating, and the population is rapidly expanding, typically the number of bacteria increases at an exponential rate.

As used herein a “multidrug-resistant” (or “MDR”) microorganism or bacteria is an organism that has an enhanced ability, relative to non-resistant strains, to resist distinct drugs or chemicals (of a wide variety of structure and function) targeted at eradicating the organism. Typically, the term refers to resistance to at least 3 classes of antibiotics.

Chemokines are small proteins secreted by cells that have the ability to induce directed chemotaxis in responsive cells. As used herein the term “interferon-inducible (ELR⁻) CXC chemokine” refers to a chemokine protein, or corresponding peptidomimetic, having a motif of four conserved cysteine residues, the first two of which are separated by a non-conserved amino acid (thus constituting the Cys-X-Cys or ‘CXC’ motif; see FIG. 1) and devoid of a three amino acid sequence, glutamic acid-leucine-arginine (the ‘ELR’ motif), immediately proximal to the CXC sequence. Examples of interferon-inducible (ELR⁻)CXC chemokines include human CXCL9, murine CXCL9, human CXCL10 (SEQ ID NO: 1), murine CXCL10, human CXCL11, and murine CXCL11. CXCL9, CXCL10, and CXCL11 are potently induced by both type 1 and type 2 interferons (IFN-α/β and IFN-γ, respectively).

As used herein, the term “adjuvant” as used herein refers to an agent which enhances the pharmaceutical effect of another agent.

The expression “amino acid” as used herein is meant to include both natural and synthetic amino acids, and both D- and L-amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the invention.

The term “amino acid” is used interchangeably with “amino acid residue” and can refer to a free amino acid and to an amino acid residue of a peptide. It will be apparent from the context in which the term is used whether it refers to a free amino acid or a residue of a peptide.

The term “antibody”, as used herein, refers to an immunoglobulin molecule which is able to specifically bind to a specific epitope on an antigen. Antibodies can be derived from natural sources or from recombinant sources and can be intact immunoglobulins or immunoreactive portions of intact immunoglobulins (for example, a fragment or derivative of an antibody that includes an antigen-binding site or a paratope). Antibodies are typically tetramers of immunoglobulin molecules. The antibodies in the present invention can exist in a variety of forms including, for example, polyclonal antibodies, monoclonal antibodies, Fv, Fab and F(ab)₂, as well as single chain antibodies and humanized antibodies (see e.g., Bird et al., 1988; Harlow & Lane, 1988; Houston et al., 1988; Harlow & Lane, 1999; each of which is incorporated herein by reference in its entirety).

The term “synthetic antibody” as used herein refers to an antibody which is generated using recombinant DNA technology, such as, for example, an antibody expressed by a bacteriophage or a host cell. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antimicrobial agent”, as used herein, refers to any entity that exhibits antimicrobial activity, i.e. the ability to inhibit the growth of and/or kill bacteria, including for example the ability to inhibit growth or reduce viability of bacteria by at least about 30%, at least about 40%, at least about 50%, at least about 60%, at least about 70% or more than 70%, as compared to bacteria not exposed to the antimicrobial agent. The antimicrobial agent can exert its effect either directly or indirectly and can be selected from a library of diverse compounds, including for example antibiotics. For example, various antimicrobial agents act, inter alia, by interfering with (1) cell wall synthesis, (2) plasma membrane integrity, (3) nucleic acid synthesis, (4) ribosomal function, and (5) folate synthesis. One of ordinary skill in the art will appreciate that a number of “antimicrobial susceptibility” tests can be used to determine the efficacy of a candidate antimicrobial agent.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (see e.g., U.S. Pat. No. 5,034,506 to Summerton Seller; Nielsen et al., 1991). The term “antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences.

As used herein, the term “biologically active fragments” or “bioactive fragment” of a polypeptide encompasses natural or synthetic portions of the full-length protein that are capable of specific binding to their natural ligand or of performing the function of the protein.

A “pathogenic” cell is a cell which, when present in a tissue, causes or contributes to a disease or disorder in the animal in which the tissue is located (or from which the tissue was obtained).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T”, is complementary to the sequence “T-C-A.”

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, the term “cytokine” refers to an intercellular signaling molecule, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that can be produced from another compound of similar structure in one or more steps, as in replacement of H by an alkyl, acyl, or amino group. Similarly, a “derivative” of a peptide (or of a polypeptide) is a compound that can be produced from or has a biological activity similar to a peptide (or a polypeptide) but that differs in the primary amino acid sequence of the peptide (or the polypeptide) to some degree. By way of example and not limitation, a derivative of a subject peptide of the presently disclosed subject matter is a peptide that has a similar although not identical primary amino acid sequence as the subject peptide (for example, has one or more amino acid substitutions) and/or that has one or more other modifications (e.g., N-terminal, C-terminal, and/or internal modifications) as compared to the subject peptide. Thus, the term “derivative” compasses the term “modified peptide” and vice versa, in the context of peptides. In some embodiments, a derivative of a peptide is a peptide that has one or more moieties conjugated thereto, including but not limited to the modifications exemplified in Table 3 below.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid of the amino acid sequence, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool; Altschul et al., 1990) are available for determining sequence identity.

In some embodiments, “identity” can be expressed as a “percent identity”. As used herein, the phrase “percent identity” in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100/nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981; by the homology alignment algorithm disclosed in Needleman & Wunsch, 1970; by the search for similarity method disclosed in Pearson & Lipman, 1988; by computerized implementations of these algorithms (GAP, BESTFIT, FASTA, and TFASTA in the GCG® WISCONSIN PACKAGE®, available from Accelrys, Inc., San Diego, California, United States of America), or by visual inspection. See generally, Altschul et al., 1990; Ausubel et al., 2002; and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff& Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

As used herein, the term “inhibit” refers to the ability of a compound or any agent to reduce or impede a described function or pathway. For example, inhibition can be in some embodiments by at least 10%, in some embodiments by at least 25%, in some embodiments by at least 50%, in some embodiments by at least 75%, in some embodiments by at least 80%, in some embodiments by at least 85%, in some embodiments by at least 90%, in some embodiments by at least 95%, in some embodiments by at least 97%, in some embodiments by at least 99%, and in some embodiments by greater than 99%.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound invention or be shipped together with a container which contains the identified compound. Alternatively, the instructional material can be shipped separately from the container with the intention that the instructional material and the compound be used cooperatively by the recipient.

An “isolated” compound/moiety is a compound/moiety that has been removed from components naturally associated with the compound/moiety. For example, an “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “modulate” refers to changing the level of an activity, function, or process. The term “modulate” encompasses both inhibiting and stimulating an activity, function, or process.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides (e.g., a polypeptide of in some embodiments at least 50 amino acids, in some embodiments at least 75 amino acids, in some embodiments at least 100 amino acids, in some embodiments at least 200 amino acids, in some embodiments at least 300 amino acids, in some embodiments at least 500 amino acids, and in some embodiments more than 500 amino acids).

A peptide encompasses a sequence of 2 or more amino acids wherein the amino acids are naturally occurring or synthetic (non-naturally occurring) amino acids.

The term “linked” or like terms refers to a connection between two entities. The linkage can comprise a covalent, ionic, or hydrogen bond or other interaction that binds two compounds or substances to one another.

As used herein the term “peptidomimetic” refers to a chemical compound having a structure that is different from the general structure of an existing peptide, but that functions in a manner similar to the existing peptide, e.g., by mimicking the biological activity of that peptide. The term “modified peptide” encompasses a peptidomimetic. Peptidomimetics typically comprise naturally occurring amino acids and/or unnatural amino acids, but can also comprise modifications to the peptide backbone. For example, a peptidomimetic can include one or more of the following modifications:
1. Peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C₁-C₆ alkyl;
2. Peptides wherein the N-terminus is derivatized to a —NRR1 group, to a —NRC(O)R group, to a —NRC(O)OR group, to a —NRS(O)₂R group, to a —NHC(O)NHR group where R and R1 are hydrogen or C₁-C₆ alkyl with the proviso that R and R1 are not both hydrogen;
3. Peptides wherein the C terminus is derivatized to —C(O)R2 where R2 is selected from the group consisting of C₁-C₆ alkoxy, and —NR3R4 where R3 and R4 are independently selected from the group consisting of hydrogen and C₁-C₄ alkyl;
4. Modification of a sequence of naturally occurring amino acids with the insertion or substitution of a non-peptide moiety, e.g., a retroinverso fragment.

The term “permeability”, as used herein, refers to transit of fluid, cell, or debris between or through cells and tissues.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds”, as used herein, is meant a compound which recognizes and binds a specific protein, but does not substantially recognize or bind other molecules in a sample, or it means binding between two or more proteins as in part of a cellular regulatory process, where said proteins do not substantially recognize or bind other proteins in a sample.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse, and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein an “amino acid modification” refers in some embodiments to a substitution, addition, or deletion of an amino acid, and includes substitution with, or addition of, any of the 20 amino acids commonly found in human proteins, as well as unusual or non-naturally occurring amino acids such as but not limited to D-amino acids. Commercial sources of unusual amino acids include Sigma-Aldrich (Milwaukee, Wisconsin, United States of America), ChemPep Inc. (Miami, Florida, United States of America), and Genzyme Pharmaceuticals (Cambridge, Massachusetts, United States of America). Unusual amino acids can be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid. The term “modified peptide” encompasses any amino acid modification as described herein.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substitutions can be designed based on, for example, the model of Dayhoff et al., 1978.

In some embodiments, an amino acid substitution is a conservative amino acid substitution. As used herein, the term “conservative amino acid substitution” is defined in some embodiments as exchanges within one of the following five groups:
I. Small aliphatic, nonpolar, or slightly polar residues: Ala, Ser, Thr, Pro, Gly;
II. Polar, charged residues and their amides: Asp, Asn, Glu, Gln, His, Arg, Lys;
III. Large, aliphatic, nonpolar residues: Met Leu, Ile, Val, Cys
IV. Large, aromatic residues: Phe, Tyr, Trp

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., 1990.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2), leucine (+3.8), phenylalanine (+2.8), cysteine/cystine (+2.5), methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet, or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. By way of example and not limitation, the following substitutions can be made: arginine and lysine; glutamate and aspartate; serine and threonine; glutamine and asparagine; and valine, leucine, and isoleucine. Alternatively, Table 1 lists exemplary conservative amino acid substitutions.

TABLE 1
Exemplary Conservative Amino Acid Substitutions

| Amino Acid | Possible Substitution(s) |
|---|---|
| Ala (A) | Leu, Ile, Val |
| Arg (R) | Gln, Asn, Lys |
| Asn (N) | His, Asp, Lys, Arg, Gln |
| Asp (D) | Asn, Glu |
| Cys (C) | Ala, Ser |
| Gln (Q) | Glu, Asn |
| Glu (E) | Gln, Asp |
| Gly (G) | Ala |
| His (H) | Asn, Gln, Lys, Arg |
| Ile (I) | Val, Met, Ala, Phe, Leu |
| Leu (L) | Val, Met, Ala, Phe, Ile |
| Lys (K) | Gln, Asn, Arg |
| Met (M) | Phe, Ile, Leu |
| Phe (F) | Leu, Val, Ile, Ala, Tyr |
| Pro (P) | Ala |
| Ser (S) | Thr |
| Thr (T) | Ser |
| Trp (W) | Phe, Tyr |
| Tyr (Y) | Trp, Phe, Thr, Ser |
| Val (V) | Ile, Leu, Met, Phe, Ala |

In some embodiments, another consideration for amino acid substitutions include whether or not the residue is located in the interior of a protein or is solvent exposed. For interior residues, conservative substitutions can include in some embodiments. Asp and Asn; Ser and Thr; Ser and Ala; Thr and Ala; Ala and Gly; Ile and Val; Val and Leu; Leu and Ile; Leu and Met; Phe and Tyr; Tyr and Trp. For solvent exposed residues, conservative substitutions can include in some embodiments: Asp and Asn; Asp and Glu; Glu and Gin; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, the Dayhoff matrix, the Grantham matrix, the McLachlan matrix, the Doolittle matrix, the Henikoff matrix, the Miyata matrix, the Fitch matrix, the Jones matrix, the Rao matrix, the Levin matrix, and the Risler matrix (summarized in, for example, Johnson & Overington, 1993; see also the PROWL resource available at the website of The Rockefeller University, New York, New York, United States of America).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### III. Compositions

### III.A. Peptides, Modified Peptides, Conjugates, and Polymers Thereof

In some embodiments, the presently disclosed subject matter relates to peptides, modified peptides, conjugates thereof, polymers thereof, and combinations thereof that have antimicrobial activity. The peptides of the presently disclosed subject matter were initially identified by consideration of the structure of the human CXCL10 (hCXCL10) gene product. CXCL10 is a small, 10 kiloDalton (kDa) protein that is produced by a variety of cells. The basic structure of hCXCL10 polypeptide is depicted in FIG. 1.

As shown in FIG. 1, hCXCL10 is characterized by several domains, including an N-terminus that is involved with interacting with the CXCR3 receptor to regulate chemotaxis and other immunomodulatory effects and a C-terminus that broadly resembles antimicrobial peptides in that it comprises a cationic amphipathic helix structure. However, it has been determined that the N-terminus also has unexpected antimicrobial activity. Between the N-terminus and the C-terminus there are three p sheets as well as the CXC domain, the two cysteines of which create cysteine-cysteine disulfide bridges, the first with a cysteine present between the first and second 0 sheets and the second with a cysteine present just N-terminal to the a helix present in the C-terminal domain. hCXC10 itself has potent activity in recruiting immune cells to sites of inflammation by virtue of its N-terminal domain interacting with CXCR3, as well as other bioregulatory activities. Full-length hCXCL10 also has direct antimicrobial activity against a variety of pathogens such as but not limited to Bacillus anthracis, Acinetobacter baumannii, and New Delhi metallo-β-lactamase (NDM) producing Klebsiella pneumoniae.

As disclosed herein, peptides derived from the N-terminus of hCXCL10 have been identified as having antimicrobial activity, including bactericidal activity. Thus, in some embodiments the presently disclosed subject matter relates to peptides comprising, consisting essentially of, or consisting of the amino acid sequence RTVRCTCI (SEQ ID NO: 19), or modified peptides thereof, or fragments thereof. Exemplary such peptides include peptides comprising, consisting essentially of, or consisting of the amino acid sequence LSRTVRCTCISI (SEQ ID NO: 11) and VPLSRTVRCTCISI (SEQ ID NO: 2), or modified peptides thereof, or fragments thereof.

Additionally, alanine scanning and truncation experiments indicated that various amino acid positions in these peptides could be substituted with other amino acids. For example, amino acids 1, 2, and 6 of RTVRCTCI (SEQ ID NO: 19) can be substituted with other amino acids and/or otherwise modified as described herein. Such modifications can include in some embodiments amino acid substitutions, which in some embodiments can be conservative amino acid substitutions, in some embodiments substitutions with unnatural amino acids, D-amino acids, and/or peptidomimetics, or any combination thereof.

In some embodiments, one, two, three, four, five, six, seven, eight, nine, ten, or more amino acids of a peptide of the presently disclosed subject matter are D-amino acids, and in some embodiments all of the amino acids of a peptide of the presently disclosed subject matter are D-amino acids. Acceptable amino acid substitutions are those that do not negatively affect the anti-bacterial ability of the D-amino acid-containing peptide. A peptide having an identical amino acid sequence to that found within a parent peptide but in which all L-amino acids have been substituted with all D-amino acids is also referred to as an “inverso” compounds. For example, if a parent peptide is Arg-Thr-Val, the inverso form is D-Arg-D-Thr-D-Val.

By way of example and not limitation, the amino acid sequence RTVRCTCI (SEQ ID NO: 19) is referred to herein as Peptide L8 or just L8. When each amino acid of SEQ ID NO: 19 is a D-amino acid, the peptide is the inverso form of Peptide L8, and is referred to herein as Peptide D8 or just D8.

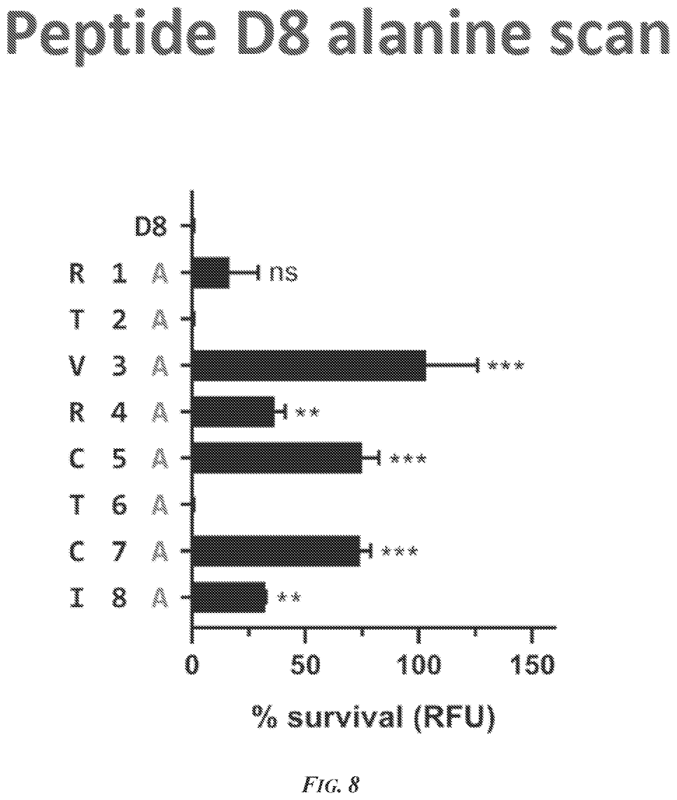

In some embodiments, the malleable positions within the presently disclosed sequences were determined by testing the activity of a series of peptide sequences in which alanine is substituted for each residue (referred to herein as “alanine scanning”). By way of example and not limitation, alanine scanning of the amino acid sequence RTVRCTCI (SEQ ID NO: 19) generates peptides with the amino acid sequences as set forth in SEQ ID NOs: 22-29: e.g., ATVRCTCI (position 1; SEQ ID NO: 22), RAVRCTCI (position 2; SEQ ID NO: 23), etc. The alanine scan establishes the malleability of these positions in more than just a conservative way (e.g. in positions 1 and 2, alanine is quite different from highly charged arginine and hydroxyl-bearing threonine, but still functional and thus included as a modified peptide in accordance with the presently disclosed subject matter). See FIG. 8.

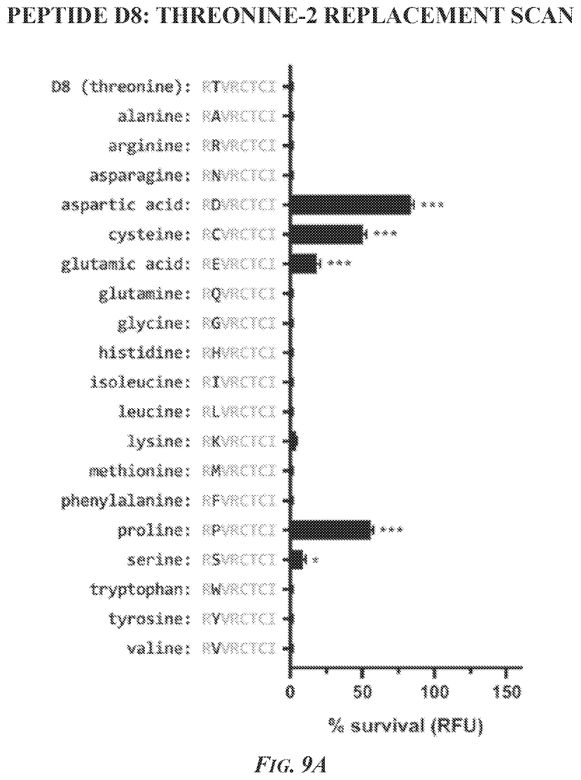

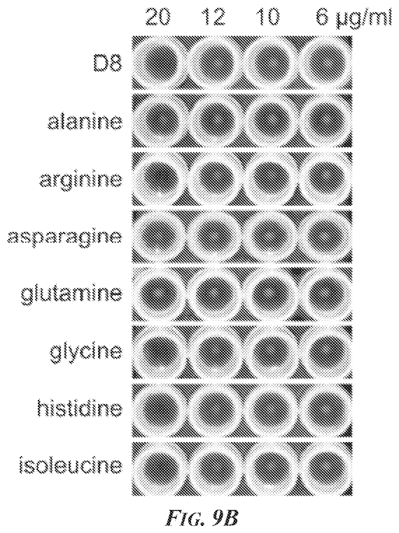

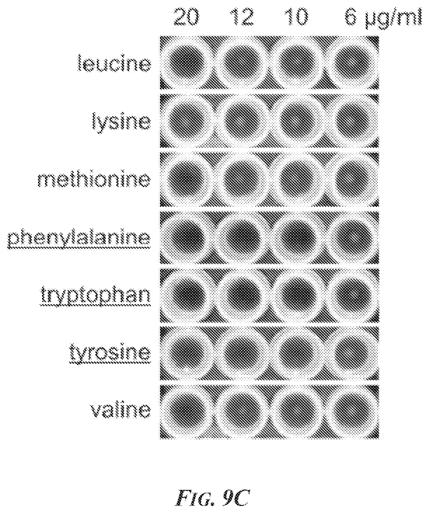

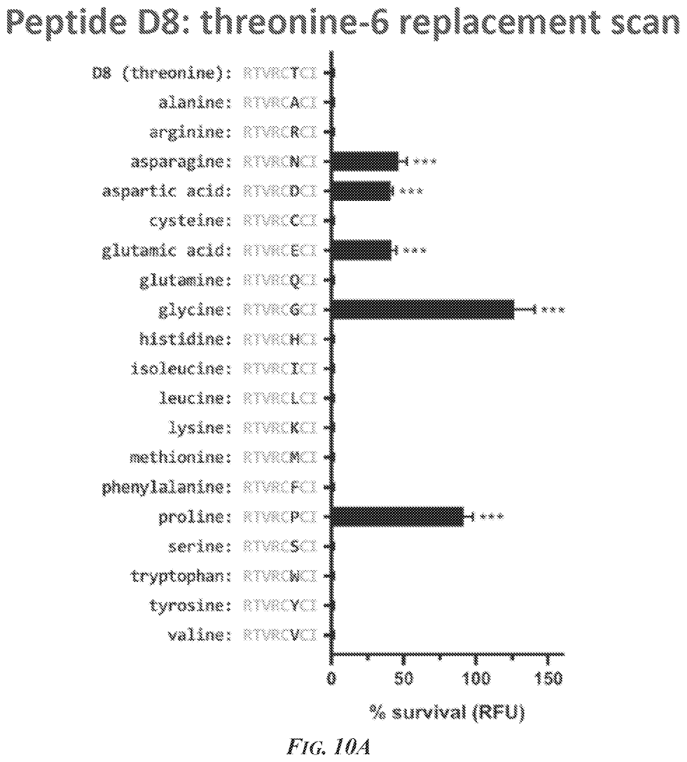

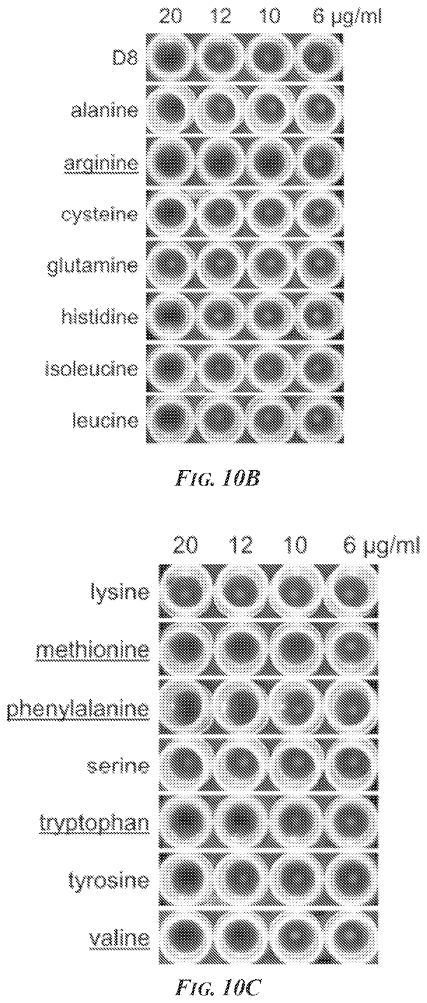

For Peptide D8 (SEQ ID NO: 19 with all D-amino acids), a replacement scan (FIG. 9A) substituted the threonine at position 2 with other D-amino acids (see SEQ ID NOs: 23 and 30-47). Also, for Peptide D8 (SEQ ID NO: 19 with all D-amino acids), a replacement scan (FIG. 10A) substituted the threonine at position 6 with other D-amino acids (see SEQ ID NOs: 27 and 48-65). Characterization of specific substitutions at other positions and in other peptides provides substitutions, or combinations thereof, that enhance peptide activity or advantageous pharmaceutical properties (e.g. solubility, stability, etc.).

In some embodiments, a peptide of the presently disclosed subject matter is a retro-inverso isomer of another peptide. As used herein, the term “retro-inverso isomer” refers a peptide in which the sequence of the amino acids is reversed as compared to the sequence of another peptide and all L-amino acids are replaced with D-amino acids. For example, if a parent peptide is Arg-Thr-Val, the retro-inverso form is DVal-DThr-DArg. Compared to the parent peptide, a retro-inverso peptide has a reversed backbone while retaining substantially the original spatial conformation of the side chains, resulting in a retro-inverso isomer with a topology that closely resembles the parent peptide (see Goodman el al., 1981. See also U.S. Pat. No. 4,522,752 for a further description of retro-inverso peptides.

In some embodiments, a peptide of the presently disclosed subject matter is a modified peptide that comprises a modification at the N-terminus, the C-terminus, or both the N-terminus and the C-terminus. In some embodiments, wherein the N-terminal and/or the C-terminal modification is selected from the group consisting of an addition or an aminohexanoic acid (AHX), an azido alanine (Ala(N₃)), a benzoylbenzoic acid (4-BBA), an azido phenylalanine (Phe(4-N₃)), a propargylglycine (PRA), or diazirine-containing amino acid residues, and/or is a replacement of a phenylalanine with an azido phenylalanine (Phe(4-N₃)). In some embodiments, the modification at the N-terminus and/or the C-terminus comprises insertion of a chemically-modified and/or unnatural amino acid at or near (e.g., within 1, 2, 3, 4, 5, or 6 amino acids) of the N- and/or C-terminus of a peptide of the presently disclosed subject matter. In some embodiments, the peptide that is modified is selected from the group consisting of SEQ ID NOs: 19, 20, 40, 42, and 58.

In some embodiments, the peptides of the presently disclosed subject matter have bactericidal and/or bacteriostatic activity against various bacteria. Exemplary bacteria for which the presently disclosed peptides have antibacterial activity include, but are not limited to Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to serovars of Salmonella enterica, including but not limited to Salmonella enterica serovar Typhi, and Shigella flexneri; and biothreat agents such as but not limited to Bacillus anthracis in both vegetative and spore forms. In some embodiments, the peptides of the presently disclosed subject matter have antibacterial activity against a multidrug-resistant (MDR) strain of a given bacterium. Exemplary non-limiting MDR bacteria for which the presently disclosed peptides have antibacterial activity include MDR strains of Enterococcus faecium, Staphylococcus aureus, Klebsiella pneumoniae, Acinetobacter baumannii, Pseudomonas aeruginosa, Salmonella enterica, optionally MDR serovars of Salmonella enterica such as but not limited to Salmonella enterica serovar Typhi, and Shigella flexneri.

The presently disclosed subject matter also provides in some embodiments conjugates and polymers comprising one or more of the peptides of the presently disclosed subject matter conjugated to a second active agent. In some embodiments, the second active agent is itself a peptide of the presently disclosed subject matter. By way of example and not limitation, a conjugate peptide of the presently disclosed subject matter can comprise, consist essentially of, or consist of conjugates between the amino acid sequence RTVRCTCI (SEQ ID NO: 19), RFVRCTCI (SEQ ID NO: 42), RWVRCTCI (SEQ ID NO: 45), RYVRCTCI (SEQ ID NO: 46), RTVRCRCI (SEQ ID NO: 48), RTVRCMCI (SEQ ID NO: 59), RYVRCRCI (SEQ ID NO: 70), or RRVRCRCI (SEQ ID NO: 72), or modified peptides thereof, or fragments thereof.

In some embodiments, the first peptide is directly conjugated to the second peptide or the first and second peptides are indirectly conjugated to each other via a linker. In some embodiments, the linker is a peptide linker, optionally a peptide of 1-9 amino acids, further optionally wherein the 1-9 amino acids are each individually selected from the group consisting of glycine and serine.

In some embodiments, a conjugate or polymer of the presently disclosed subject matter comprises one or more peptides of the presently disclosed subject matter, or one or more modified peptides and/or fragments thereof, or any combination thereof, wherein each peptide present in the conjugate is covalently linked to at least one other peptide via a non-peptide linker, a peptide linker, or a cysteine-cysteine linkage. In some embodiments, the conjugate or polymer comprises a branched conjugate, a flanking conjugate, a single conjugate, a linear polymer, a bottlebrush polymer, or any combination thereof.

In some embodiments, pegylation is possible at the N-terminus, or even by incorporating non-natural amino acids with PEGylated side chains (such non-natural amino acids incorporated into peptides).

In some embodiments, other modifications are provided. By way of example and not limitation, dyes are incorporated on peptide side chains or at the chain end, such as to facilitate pharmacokinetic studies. Such dyes range from rhodamine B (red) and fluorescein (green) to near-IR dyes that are compatible with animal imaging. Alternatively or in addition, peptide side chains, chain ends, and/or other sites can be modified to comprise one or more moieties that can be detected by imaging techniques including but not limited to photoacoustic imaging (PAI) and/or Positron Emission Tomography (PET), such as but not limited to for the purpose of tracking a peptide in vivo. In some embodiments, an isotopically-labeled amino acid residue can be incorporated into an individual amino acid or amino acid chain for PET scanning, including but not limited to ⁶⁴Cu, ¹²⁴I, ^{76/77}Br, ⁸⁶Y, ⁸⁹Zr, ⁶⁸Ga, ¹⁸F, ¹¹C, ¹²⁵I, ¹²⁴I, ¹³¹I, ¹²³I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ¹⁷⁷Lu, ⁹⁹Tc, and ¹⁵³Sm.

In some embodiments of a modified peptide in accordance with the presently disclosed subject matter, cysteine is replaced with selenocysteine: In some embodiments, conservative substitutions, like threonine to serine and valine to leucine/isoleucine are employed, but also substitutions with dyes are provided in the malleable positions to allow evaluation of in vivo half-life, biodistribution, etc. An example of a dye-modified residue is a lysine residue where the amine side chain is functionalized with rhodamine B (bearing a carboxylic acid, therefore the same chemistry used to couple amino acids together is used on this side chain reaction). In some embodiments, cysteine residues are capped to abolish their reactivity and also tested for a balance of effects on peptide stability and function.

In some embodiments, a conjugate or polymer of the presently disclosed subject matter is polymer-functionalized, encapsulated in a particle, embedded in and/or on a solid support, impregnated on a dressing, and/or is formulated for use in a nebulizer, for topical administration, and/or for systemic administration. In some embodiments, the conjugate that is embedded/immobilized in and/or on a solid support such as a surface of a medical device such as a stent and/or impregnated on a dressing can be released from the solid support and/or the dressing, optionally wherein the release occurs when the solid support and/or the dressing comes in contact with a subject, optionally a bodily fluid, cell, tissue, or organ of a subject. In some embodiments, the release occurs over a pre-determined time frame. In some embodiments, the solid support and/or the dressing comprises a plurality of particles, wherein each particle is associated with, conjugated to, and/or encapsulates a peptide and/or a conjugate of the presently disclosed subject matter. In some embodiments, the plurality of particles are characterized by different release profiles, at least one of which is a slow-release profile, at least one of which is a fast-release profile, or combinations thereof (see e.g., U.S. Patent Application Publication No. 2011/0218140 for examples of slow-release and fast-release nanoparticles.

Thus, in some embodiments the presently disclosed subject matter relates to a wound dressing, wherein the wound dressing comprises a support layer with an antibacterial agent embedded therein or associated therewith. In some embodiments, the antibacterial agent comprises, consists essentially of, or consists of a peptide as disclosed herein, a conjugate as disclosed herein, a polymer as disclosed herein, or any combination thereof. In some embodiments, the peptide and/or conjugate is encapsulated in one or more particles that are embedded in or associated with the support layer. In some embodiments, some or all of the one or more particles are designed to release from the support layer when the dressing is in contact with a wound, and in some embodiments some or all of the one or more particles are designed to be retained in the support layer when the dressing is in contact with a wound.

### III.B. Pharmaceutical Compositions

In some embodiments, the peptides and/or conjugates of the presently disclosed subject matter are present in a pharmaceutical composition. Thus, in some embodiments the presently disclosed subject matter relates to pharmaceutical compositions comprising, consisting essentially of, or consisting of one or more peptides as disclosed herein, one or more conjugates as disclosed herein, one or more polymers as disclosed herein, or any combination thereof, along with one or more pharmaceutically acceptable carriers, diluents, or excipients. In some embodiments, the presently disclosed pharmaceutical compositions are pharmaceutically acceptable for use in humans.

Thus, the disclosed pharmaceutical compositions can be employed by administration to a subject in need thereof. In some embodiments, the disclosed pharmaceutical compositions can be administered in vivo in a pharmaceutically acceptable carrier. By “pharmaceutically acceptable” is meant a material that is not biologically or otherwise undesirable, i.e., the material can be administered to a subject, along with a peptide composition of the presently disclosed subject matter, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution and/or in suspension (for example, incorporated into microparticles, liposomes, and/or cells.

As would be understood by those of skill in the art, when peptides are synthesized, they are typically prepared with counter-ions to stabilize basic/acidic sidechains of amino acid residues. Thus, peptide drugs often occur in the form of salts where different counter-ions can affect peptide structure, function, etc. differently. In particular, cationic peptides like those disclosed herein are typically obtained as trifluoroacetic acid (TFA) salts. However, TFA can be toxic and have off-target effects making it an unfavorable drug formulation for some uses. To address these issues, in some embodiments TFA has been exchanged with acetate or formate, each of which is non-toxic and can be used in peptide pharmaceuticals. In some embodiments, the acetate formulation of a peptide of the presently disclosed subject matter provides better bactericidal activity than the formate formulation.

The peptide, conjugate, and polymer compositions of the presently disclosed subject matter can be used therapeutically in combination with one or more pharmaceutically acceptable carriers.

Suitable carriers and their formulations are described in Remington et al., 1975. Typically, an appropriate amount of a pharmaceutically acceptable salt is used in the formulation to render the formulation isotonic. Examples of the pharmaceutically acceptable carrier include, but are not limited to, saline, Ringer's solution, and dextrose solution. The pH of the solution is in some embodiments from about 5 to about 8, and in some embodiments from about 7 to about 7.5. Further carriers include sustained release preparations such as semipermeable matrices of solid hydrophobic polymers containing the peptide compositions, which matrices are in the form of shaped articles, e.g., films, liposomes, or microparticles. It will be apparent to those persons skilled in the art that certain carriers can be selected depending upon, for instance, the route of administration and/or concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents, and the like, in addition to the molecule of choice. Pharmaceutical compositions can also include one or more active ingredients such as antimicrobial agents, anti-inflammatory agents, anesthetics, and the like.

The pharmaceutical composition can be administered in a number of ways depending on whether local or systemic treatment is desired, and on the area to be treated. Administration can occur topically (including ophthalmically, vaginally, rectally, intranasally), orally, by inhalation, or parenterally, for example by intravenous drip, subcutaneous, intraperitoneal, or intramuscular injection. The disclosed peptide compositions can be administered in some embodiments topically, intravenously, intraperitoneally, intramuscularly, subcutaneously, intracavity, or transdermally.

Preparations for parenteral administration can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like.

Formulations for topical administration can include ointments, lotions, creams, gels, drops, suppositories, sprays, liquids, and powders. Conventional pharmaceutical carriers, aqueous, powder or oily bases, thickeners, and the like can also be employed, as desired.

Compositions for oral administration include powders or granules, suspensions or solutions in water or non-aqueous media, capsules, sachets, or tablets. Thickeners, flavorings, diluents, emulsifiers, dispersing aids, or binders can in some embodiments also be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, or phosphoric acid and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl, and aryl amines and substituted ethanolamines.

### IV. Methods for Using the Peptides, Modified Peptides, and Conjugates of the Presently Disclosed Subject Matter

The presently disclosed subject matter compositions and pharmaceutical compositions can be employed for preventing and/or treating microorganismal infections either in vivo, ex vivo, or in vitro. Thus, in some embodiments the presently disclosed subject matter relates to methods for inhibiting the growth of and/or killing a bacterium. In some embodiments, the methods comprise contacting the bacterium with an effective amount of an antibacterial agent, wherein the antibacterial agent comprises, consists essentially of, or consists of a peptide as disclosed herein, a conjugate as disclosed herein, a polymer as disclosed herein, or any combination thereof.

Infection with both Gram-negative and Gram-positive bacteria can be treated and/or prevented using the compositions and methods of the presently disclosed subject matter. By way of example and not limitation, in some embodiments the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to serovars of Salmonella enterica, including but not limited to Salmonella enterica serovar Typhi, and Shigella flexneri; and biothreat agents such as but not limited to Bacillus anthracis in both vegetative and spore forms. In some embodiments, the bacterium is selected from the group consisting of Enterococcus spp. including but not limited to Enterococcus faecium such as but not limited to vancomycin-resistant E. faecium (VRE), Staphylococcus aureus including but not limited to methicillin-resistant S. aureus (MRSA), Klebsiella pneumoniae including but not limited to multidrug resistant and carbapenem-resistant K. pneumoniae, Acinetobacter spp. including but not limited to multidrug-resistant Acinetobacter spp., Pseudomonas aeruginosa including but not limited to multidrug-resistant P. aeruginosa, Enterobacteriaceae including but not limited to MDR and/or CRE Escherichia coli and Klebsiella spp., enteric pathogens including but not limited to multidrug-resistant Salmonella enterica serovars such as serovar Typhi and multidrug-resistant Shigella flexneri, sexually-transmitted bacteria such as but not limited to Neisseria gonorrhoeae, and biothreat agents such as but not limited to both the vegetative and spore forms of Bacillus anthracis.

In some embodiments, the presently disclosed subject matter also relates to methods for treating bacterial infections present in wounds. In some embodiments, the methods comprise contacting the wound with an effective amount of a composition comprising one or more peptides, each peptide comprising, consisting essentially of, or consisting of an amino acid sequence as set forth in SEQ ID NOs: 2-151, or modified peptides thereof, or fragments thereof. In some embodiments, composition comprising one or more peptides, each peptide comprising, consisting essentially of, or consisting of an amino acid sequence as set forth in SEQ ID NOs: 2-151, or modified peptides thereof, or fragments thereof.

In some embodiments, an infection can be a pulmonary infection, and thus the presently disclosed subject matter related in some embodiments to methods for treating pulmonary infections in subjects by administering to a subject in need thereof an effective amount of a composition comprising one or more peptides, conjugates, and/or polymers, each peptide and/or conjugate and/or polymer comprising, consisting essentially of, or consisting of an amino acid sequence as set forth in SEQ ID NOs: 2-151, or modified peptides thereof, or fragments thereof. In some embodiments, the composition is administered to the subject intranasally, by inhalation, optionally wherein the one or more peptides in the composition is/are aerosolized, or any combination thereof.

The presently disclosed compositions can also be employed for treating or preventing systemic bacterial infections in subjects. In some embodiments, the methods comprise administering to a subject in need thereof an effective amount of a composition comprising one or more peptides and/or conjugates and/or polymers, each peptide and/or conjugate and/or polymer comprising, consisting essentially of, or consisting of an amino acid sequence as set forth in SEQ ID NOs: 2-151, or modified peptides thereof, or fragments thereof. The presently disclosed compositions can also be employed in a combination therapy in which the composition comprising one or more peptides and/or conjugates comprising, consisting essentially of, or consisting of an amino acid sequence as set forth in SEQ ID NOs: 2-151, or modified peptides thereof, or fragments thereof, is administered to the subject before, after, or concurrently with a second antibacterial therapy, which in some embodiments can involve the use of a conventional antibiotic. In some embodiments, the conventional antibiotic is selected from the group consisting of penicillins, cephalosporins, carbepenems, other beta-lactams antibiotics, aminoglycosides, macrolides, lincosamides, glycopeptides, tetracylines, chloramphenicol, quinolones, fucidins, sulfonamides, triinethoprims, rifamycins, oxalines, streptogramins, lipopeptides, ketolides, polyenes, azoles, and echinocandins.

Thus, in some embodiments the presently disclosed subject matter relates to any and all uses of the peptides disclosed herein, the conjugates disclosed herein, or any combination thereof for preventing or treating a bacterial infection.

Given that antibacterial activity of the presently disclosed subject matter peptides, conjugates, and/or polymers, the presently disclosed subject matter also provides in some embodiments methods for treating or preventing community and/or nosocomial infections in subjects. In some embodiments, the methods comprise administering to a subject at risk for developing and/or who has developed a community and/or nosocomial infection a composition of the presently disclosed subject matter. In some embodiments, the composition comprises a peptide comprising, consisting essentially of, or consisting of an amino acid sequence as set forth in any of SEQ ID NOs: 2-151, a modified peptide thereof, a fragment thereof, or a combination thereof. SEQ ID NOs: 2-151 are set forth in Table 2.

TABLE 2
Exemplary Base Sequences of the Presently
Disclosed Subject Matter

| SEQ ID | |
|---|---|
| NO: | SEQUENCE |
| 1 | VPLSRTVRCTCISISNQPVNPRSLEKLEIIPASQFCPRVE |
| | IIATMKKKGEKRCLNPESKAIKNLLKAVSKERSKRSP |
| 2 | VPLSRTVRCTCISI |
| 3 | TCISISNQPVNPRSL |
| 4 | NPRSLEKLEIIPASQ |
| 5 | IPASQFCPRVEIIAT |
| 6 | EIIATMKKKGEKRCL |
| 7 | EKRCLNPESKAIKNL |
| 8 | KNLLKAVSKERSKRSP |
| 9 | PESKAIKNLLKAVSKERSKRSP |
| 10 | PLSRTVRCTCISI |
| 11 | LSRTVRCTCISI |
| 12 | SRTVRCTCISI |
| 13 | RTVRCTCISI |
| 14 | TVRCTCISI |
| 15 | VRCTCISI |
| 16 | RCTCISI |
| 17 | CTCISI |
| 18 | RTVRCTCIS |
| 19 | RTVRCTCI |
| 20 | RTVRCTC |
| 21 | RTVRCT |
| 22 | ATVRCTCI |
| 23 | RAVRCTCI |
| 24 | RTARCTCI |
| 25 | RTVACTCI |
| 26 | RTVRATCI |
| 27 | RTVRCACI |
| 28 | RTVRCTAI |
| 29 | RTVRCTCA |
| 30 | RRVRCTCI |
| 31 | RNVRCTCI |
| 32 | RDVRCTCI |
| 33 | RCVRCTCI |
| 34 | REVRCTCI |
| 35 | RQVRCTCI |
| 36 | RGVRCTCI |
| 37 | RHVRCTCI |
| 38 | RIVRCTCI |
| 39 | RLVRCTCI |
| 40 | RKVRCTCI |
| 41 | RMVRCTCI |
| 42 | RFVRCTCI |
| 43 | RPVRCTCI |
| 44 | RSVRCTCI |
| 45 | RWVRCTCI |
| 46 | RYVRCTCI |
| 47 | RVVRCTCI |
| 48 | RTVRCRCI |
| 49 | RTVRONCI |
| 50 | RTVRCDCI |
| 51 | RTVRCCCI |
| 52 | RTVRCECI |
| 53 | RTVRCQCI |
| 54 | RTVRCGCI |
| 55 | RTVRCHCI |
| 56 | RTVRCICI |
| 57 | RTVRCLCI |
| 58 | RTVRCKCI |
| 59 | RTVRCMCI |
| 60 | RTVRCFCI |
| 61 | RTVRCPCI |
| 62 | RTVRCSCI |
| 63 | RTVRCWCI |
| 64 | RTVRCYCI |
| 65 | RTVRCVCI |
| 66 | RFVRCRCI |
| 67 | RFVRCMCI |
| 68 | RWVRCRCI |
| 69 | RWVRCMCI |
| 70 | RYVRCRCI |
| 71 | RYVRCMCI |
| 72 | RRVRCRCI |
| 73 | RWVRCWCI |
| 106 | AYVRCRCI |
| 107 | NYVRCRCI |
| 108 | DYVRCRCI |
| 109 | CYVRCRCI |
| 110 | QYVRCRCI |
| 111 | EYVRCRCI |
| 112 | GYVRCRCI |
| 113 | HYVRCRCI |
| 114 | IYVRCRCI |
| 115 | LYVRCRCI |
| 116 | KYVRCRCI |
| 117 | MYVRCRCI |
| 118 | FYVRCRCI |
| 119 | PYVRCRCI |
| 120 | SYVRCRCI |
| 121 | TYVRCRCI |
| 122 | WYVRCRCI |
| 123 | YYVRCRCI |
| 124 | VYVRCRCI |
| 129 | RAVRCRCI |
| 130 | RRVRCRCI |
| 131 | RNVRCRCI |
| 132 | RDVRCRCI |
| 133 | RCVRCRCI |
| 134 | RQVRCRCI |
| 135 | REVRCRCI |
| 136 | RGVRCRCI |
| 137 | RHVRCRCI |
| 138 | RIVRCRCI |
| 139 | RLVRCRCI |
| 140 | RKVRCRCI |
| 141 | RMVRCRCI |
| 142 | RFVRCRCI |
| 143 | RPVRCRCI |
| 144 | RSVRCRCI |
| 145 | RTVRCRCI |
| 146 | RWVRCRCI |
| 147 | RVVRCRCI |

Similarly, the presently disclosed subject matter also provides in some embodiments methods for inducing a subject's immune system against a pathogen by administering to the subject a composition comprising a peptide comprising, consisting essentially of, or consisting of an amino acid sequence as set forth in any of SEQ ID NOs: 2-151, a modified peptide thereof, a fragment thereof, or a combination thereof.

In some embodiments of the presently disclosed peptides, the N-terminal VP amino acids are required. In some embodiments, D-peptides are not expected to engage the immune system, e.g., not expected to activate the cellular receptor and induce immune-cell recruitment. Incidentally, D-peptides are also expected to be more stable—as they are less susceptible to protease degradation. In some embodiments, the peptides (either L-amino acid versions or D-amino acid versions) bind the receptor, but do not activate it. These act as inhibitors and can be employed to reduce inflammation. This is another application for CXCL10-derived peptides in accordance with the presently disclosed subject matter. Indeed, inhibition of the CXCL10-CXCR3 receptor signaling axis has been shown to be therapeutically beneficial in a number of disease states.

In some embodiments, the modified peptides include modifications of a peptide of the presently disclosed subject matter at the N-terminus, the C-terminus, or both the N-terminus and the C-terminus. In order to provide compositions for mechanistic investigations, bacterial-target identification, and microscopy/molecular imaging, labeling strategies have been developed for CXCL10-derived antimicrobial peptides that do not abolish bactericidal activity. Preliminary studies showed that pre-labeling lead-candidate peptides, for example with biotin, results in a loss of activity. To overcome this challenge, modified peptide variants were designed that include azide-, alkyne-, and/or diazirine-containing unnatural amino acids. These functional groups are not generally present in nature and they do not react innately with proteins or other biomolecules. Azide-alkyne cycloaddition reactions (e.g., click chemistry), however, can be used to facilitate site-specific bioconjugation and thereby allow effective peptide labeling to occur after interaction with a target/bacterium.

By way of example and not limitation, azide-containing azido alanine or alkyne-containing propargylglycine can be used alone for peptide labeling/recovery, as well as in combination with a photo-reactive moiety such as benzoylbenzoic acid (4-BBA), an azido phenylalanine (Phe(4-N₃)), and/or a diazirine-containing amino acid, optionally leucine, methionine, lysine, proline, or phenylalanine for enhanced functionality including target capture. Exemplary peptides of the presently disclosed subject matter modified in this fashion include the following derivatives of the peptide sequences RTVRCTCI (SEQ ID NO: 19), RTVRCTC (SEQ ID NO: 20), RKVRCTCI (SEQ ID NO: 40), RFVRCTCI (SEQ ID NO: 42), RTVRCKCI (SEQ ID NO: 58), RTVRCTCIK (SEQ ID NO: 74), FRTVRCTCI (SEQ ID NO: 75), FRRVRCTCI (SEQ ID NO: 76), RTVRCTCIA (SEQ ID NO: 77), FRTVRCTCIA (SEQ ID NO: 78), FRRVRCTCIA (SEQ ID NO: 79), FRRVRCRCIA (SEQ ID NO: 80), ARTVRCTCI (SEQ ID NO: 81), ARTVRCTCIF (SEQ ID NO: 82), ARRVRCTCIF (SEQ ID NO: 83), ARRVRCRCIF (SEQ ID NO: 84), MRTVRCTCIA (SEQ ID NO: 85), LRTVRCTCIA (SEQ ID NO. 86), KRTVRCTCIA (SEQ ID NO: 87), ARTVRCTCIM (SEQ ID NO: 88), ARTVRCTCIL (SEQ ID NO: 89), and ARTVRCTCK (SEQ ID NO: 90), although similar modifications can be made to any of the peptides of the presently disclosed subject matter. By way of example and not limitation, exemplary derivatives of the peptides of the presently disclosed subject matter are presented in Table 3.

In Table 3 below, the abbreviations are as follows: conventional one-letter codes are used to identify unmodified D-amino acids. AHX is aminohexanoic acid. Ala(N₃) is azido alanine. 4-BBA is benzoylbenzoic acid. Phe(4-N₃) is azido phenylalanine. PRA is propargylglycine. “D” and “L” in reduced font size are employed to distinguish D amino acids and L amino acids from D and L as one-letter amino acid abbreviations for aspartic acid and leucine, respectively. By way of example, “DR” refers to D-arginine whereas “DR” refers to the dipeptide aspartic acid-arginine. photo is indicative of a photoaffinity label.

TABLE 3
Exemplary Derivatives of the Presently Disclosed Peptides

| | | |
|---|---|---|
| Peptide Name | SEQ ID NO: | Sequence |

Biotin Derivatives

| | | |
|---|---|---|
| P154 | 19 | Biotin-AHX-DR-DT-DV-DR-DC-DT-DC-DI |
| P211 | 40 | DR-(DK-Biotin)-DV-DR-DC-DT-DC-DI |
| P212 | 58 | DR-DT-DV-DR-DC-(DK-Biotin)-DC-DI |
| P213 | 74 | DR-DT-DV-DR-DC-DT-DC-DI-(DK-Biotin) |

Phe(4-N₃) Derivatives

| | | |
|---|---|---|
| P214 | 42 | DR-(LF(4N₃))-DV-DR-DC-DT-DC-DI |
| P215 | 42 | DR-(DF(4N₃))-DV-DR-DC-DT-DC-DI |
| P216 | 76 | (LF(4N₃))-DR-DT-DV-DR-DC-DT-DC-DI |
| P217 | 76 | (DF(4N₃))-DR-DT-DV-DR-DC-DT-DC-DI |

Propargylglycine (PRA) Derivatives

| | | |
|---|---|---|
| P218 | 19 | DR-DT-DV-DR-DC-DT-DC-DI-(L-PRA) |
| P219 | 19 | DR-DT-DV-DR-DC-DT-DC-DI-(D-PRA) |
| P220 | 20 | DR-DT-DV-DR-DC-DT-DC-(L-PRA) |
| P221 | 20 | DR-DT-DV-DR-DC-DT-DC-(D-PRA) |

Phe(4-N₃) + PRA Derivatives

| | | |
|---|---|---|
| P227 | 76 | (DF(4N₃))-DR-DT-DV-DR-DC-DT-DC-DI-(D-PRA) |
| P228 | 42 | DR-(DF(4N₃))-DV-DR-DC-DT-DC-DI-(D-PRA) |
| P242 | 77 | (DF(4N₃))-DR-DR-DV-DR-DC-DT-DC-DI-(D-PRA) |

4-BBA + PRA Derivatives

| | | |
|---|---|---|
| P229 | 19 | (4~BBA-DR)-DT-DV-DR-DC-DT-DC-DI-(D-PRA) |
| P230 | 40 | DR-(DK-4-BBA)-DV-DR-DC-DT-DC-DI-(D-PRA) |

Ala(Na) +/− Phe(4-N₃) Derivatives

| | | |
|---|---|---|
| P244 | 78 | DR-DT-DV-DR-DC-DT-DC-DI-(DA(N3)) |
| P245 | 79 | (DF(4N₃))-DR-DT-DV-DR-DC-DT-DC-DI-(DA(N₃)) |
| P246 | 80 | (DF(4N₃))-DR-DR-DV-DR-DC-DT-DC-DI-(DA(N₃)) |
| P248 | 81 | (DF(4N₃))-DR-DR-DV-DR-DC-DR-DC-DI-(DA(N₃)) |
| P249 | 82 | (DA(N₃))-DR-DT-DV-DR-DC-DT-DC-DI |
| P250 | 83 | (DA(N₃))-DR-DT-DV-DR-DC-DT-DC-DI-(DF(4N₃)) |
| P251 | 84 | (DA(N₃))-DR-DR-DV-DR-DC-DT-DC-DI-(DF(4N₃)) |
| P252 | 85 | (DA(N₃))-DR-DR-DV-DR-DC-DR-DC-DI-(DF(4N₃)) |

Ala(N3) +/− Diazirine Met/Leu/Lys Derivatives

| | | |
|---|---|---|
| P253 | 86 | (L-photo-M)-DR-DT-DV-DR-DC-DT-DC-DI-(DA(N₃)) |
| P254 | 87 | (L-photo-L)-DR-DT-DV-DR-DC-DT-DC-DI-(DA(N₃)) |
| P255 | 88 | (L-photo-K)-DR-DT-DV-DR-DC-DT-DC-DI-(DA(N₃)) |
| P256 | 89 | (DA(N₃))-DR-DT-DV-DR-DC-DT-DC-DI-(L-photo-M) |
| P257 | 90 | (DA(N₃))-DR-DT-DV-DR-DC-DT-DC-DI-(L-photo-L) |
| P258 | 91 | (DA(N₃))-DR-DT-DV-DR-DC-DT-DC-DI-(L-photo-K) |

Ala(N3) +/− Diazirine Met/Leu/Lys Derivatives

| | | |
|---|---|---|
| P253DPRA | 92 | (L-photo-M)-DR-DT-DV-DR-DC-DT-DC-DI-(D-PRA) |
| P254DPRA | 93 | (L-photo-L)-DR-DT-DV-DR-DC-DT-DC-DI-(D-PRA) |
| P255DPRA | 94 | (L-photo-K)-DR-DT-DV-DR-DC-DT-DC-DI-(D-PRA) |
| P256DPRA | 95 | (D-PRA)-DR-DT-DV-DR-DC-DT-DC-DI-(L-photo-M) |
| P257DPRA | 96 | (D-PRA)-DR-DT-DV-DR-DC-DT-DC-DI-(L-photo-L) |
| P258DPRA | 97 | (D-PRA)-DR-DT-DV-DR-DC-DT-DC-DI-(L-photo-K) |

Miscellaneous Derivatives*

| | | |
|---|---|---|
| D8-T2alloThr | 98 | R[alloThr]VRCTCI |
| D8-T2MeThr | 99 | R[MeThr]VRCTCI |
| D8-T2Abu | 100 | R[Abu]VRCTCI |
| D8-T2NapAla | 101 | R[NapthylAla]VRCTCI |
| D8-T6alloThr | 102 | RTVRC[allo Thr]CI |
| D8-T6MeThr | 103 | RTVRC[MeThr]CI |
| D8-T6Abu | 104 | RTVRC[Abu]CI |
| D8-T6NapAla | 105 | RTVRC[NapthylAla]CI |
| P194-R1Cit | 125 | [Cit]YVRCRCI |
| P194-R1AR | 126 | [hR]YVRCRCI |
| P194-R1R(NO2) | 127 | [R(NO₂)]YVRCRCI |
| P194-R1Agb | 128 | [Agb]YVRCRCI |
| P194-Y2MeTyr | 148 | R[MeTyr]VRCRCI |
| P194-Y2blodoTyr | 149 | R[Iodo-Tyr]VRCRCI |
| P194-Y2DiMethylTyr | 150 | R[DimethylTyr]VRCRCI |
| P194-Y2bP | 151 | R[hP]VRCRCI |
| PEG2-D8 | 19* | PEG2-RTVRCTCI |
| PEG4-D8 | 19* | PEG4-RTVRCTCI |
| PEG2-P194 | 70* | PEG2-RYVRCRCI |
| PEG4-P194 | 70* | PEG4-RYVRCRCI |

*alloThr-allothreonine; MeThr-o-methyl threonine; Abu-aminobutyric; NapthylAla-ala(2naphthyl); Cit-citrulline; hR: homoarginine; R(NO2): nitroarginine; Agb: norarginine; PEG2-2 PEG moieties; PEG4-4 PEG moieties.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the Examples

Several of the methods employed are described in more detail in Crawford et al., 2023 and Wiegand et al., 2008. More particularly, Crawford et al., 2023 includes detailed methods for many of the experiments including the alamarBlue bacterial killing assay, hemolysis and cytotoxicity assays, and chemotaxis assay. All minimum inhibitory concentrations (MICs) were determined using standardized broth microdilution methodology that is described in Wiegand et al., 2008. Other details for the assays described herein (e.g., peptide concentration) are included in the corresponding Figure legends.

### Example 1

### Identification of Antimicrobial Subsequences of CXCL10

CXCL10 was known to kill bacteria, but the regions or domains responsible for this activity were not known. To determine the antibacterial domains of CXCL10, a library of overlapping peptides was prepared and tested for antimicrobial activity. The library is depicted in FIG. 1. As described herein, the N-terminus and the C-terminus were found to kill bacteria. The P1 peptide (SEQ ID NO. 2) and derivatives thereof were selected for further analysis.

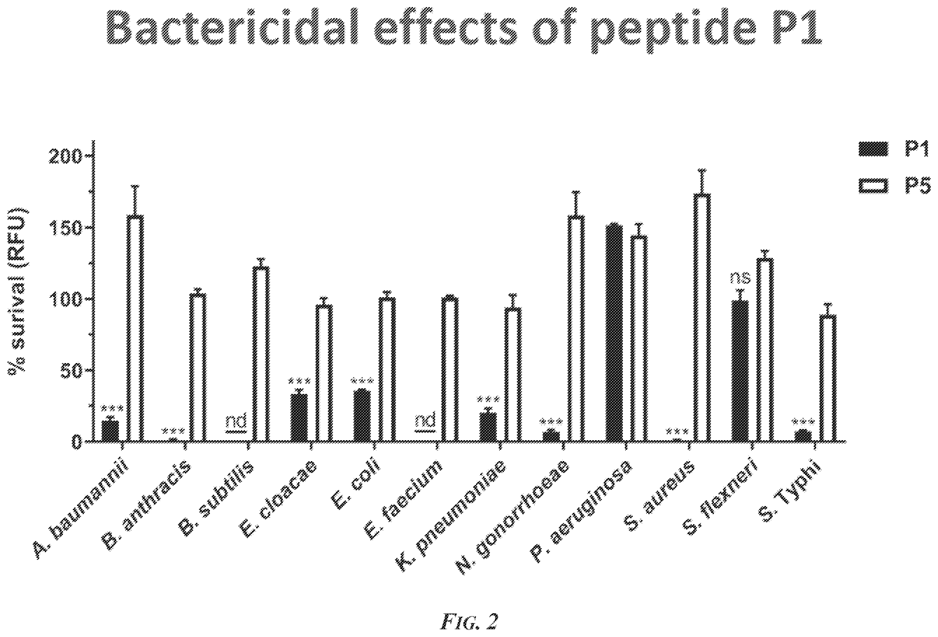

To define the spectrum of organisms susceptible to the original N-terminal peptide P1 (SEQ ID NO: 2), the viability reagent alamarBlue and a collection of Biodefense, food-borne, and antibiotic-resistant pathogens were employed. The results are shown in FIG. 2.

As shown in FIG. 2, Peptide P1 (SEQ ID NO: 2) displayed broad spectrum bactericidal activity against Gram-positive and Gram-negative organisms including Acinetobacter baumannii, Bacillus anthracis, Bacillus subtilis, Enterobacter cloacae, Escherichia coli, Enterococcus faecium, Klebsiella pneumoniae, Neisseria gonorrhoeae, Staphylococcus aureus, and Salmonella enterica serovar Typhi.

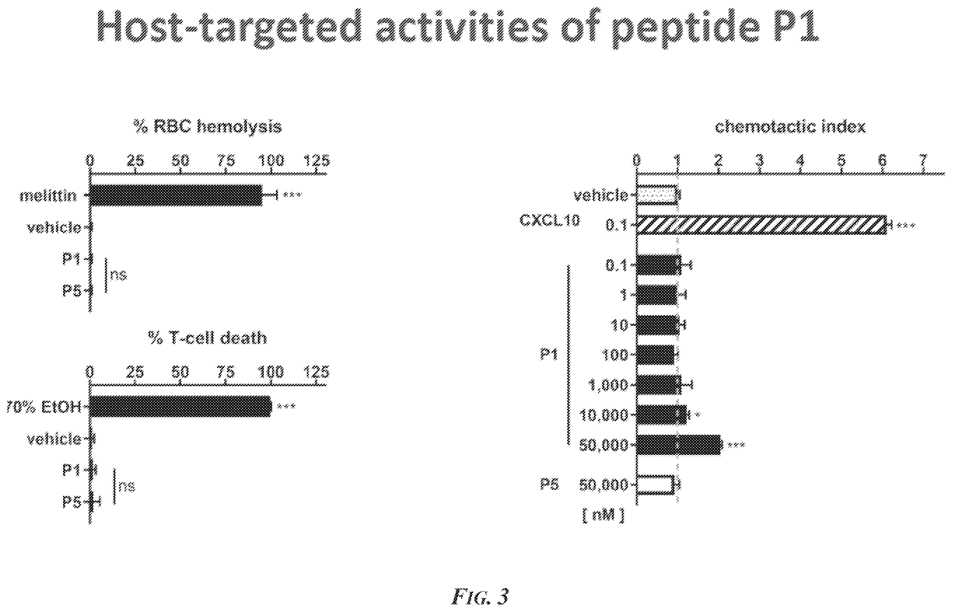

To test the ability of Peptide P1 (SEQ ID NO: 2) to kill eukaryotic cells, it was tested for hemolytic and cytotoxic effects. Since Peptide P1 (SEQ ID NO: 2) is derived from a region of CXCL10 that is important for binding the cellular receptor that elicits chemotaxis, whether this host-targeted effect was retained was tested. The results are shown in FIG. 3.

As shown in FIG. 3, Peptide P1 (SEQ ID NO: 2) did not lyse human red blood cells or kill human T cells (see FIG. 3, left panel). At concentrations that effectively killed various bacteria, Peptide P1 (SEQ ID NO: 2) was also shown to cause T cell chemotaxis through the CXCR3 receptor (see FIG. 3, right panel).

### Example 2

### Biological Activities of Derivatives of Peptide P1 (SEO ID NO: 2)

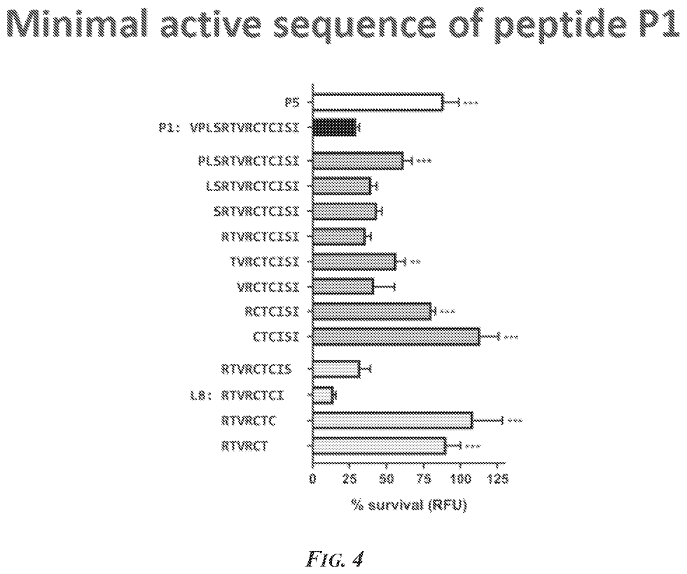

Peptide P1 (SEQ ID NO: 2) had antimicrobial activity, so what the actual minimal active sequence might be was tested. To do this, truncation mutants were synthesized by removing one amino acid at a time from the N-terminus and then from the C-terminus of Peptide P1 (SEQ ID NO: 2) until activity was lost as determined using alamarBlue. The results are shown in FIG. 4.

As shown in FIG. 4, the minimal active sequence of the N-terminal antimicrobial domain of CXCL10 is an 8-mer sequence referred to herein as Peptide L8 (RTVRCTCI; SEQ ID NO: 19). This Peptide when then used as a starting point for further optimization and development.

### Example 3

### Biological Activities of D-Amino Acid Derivatives of SEO ID NO: 19

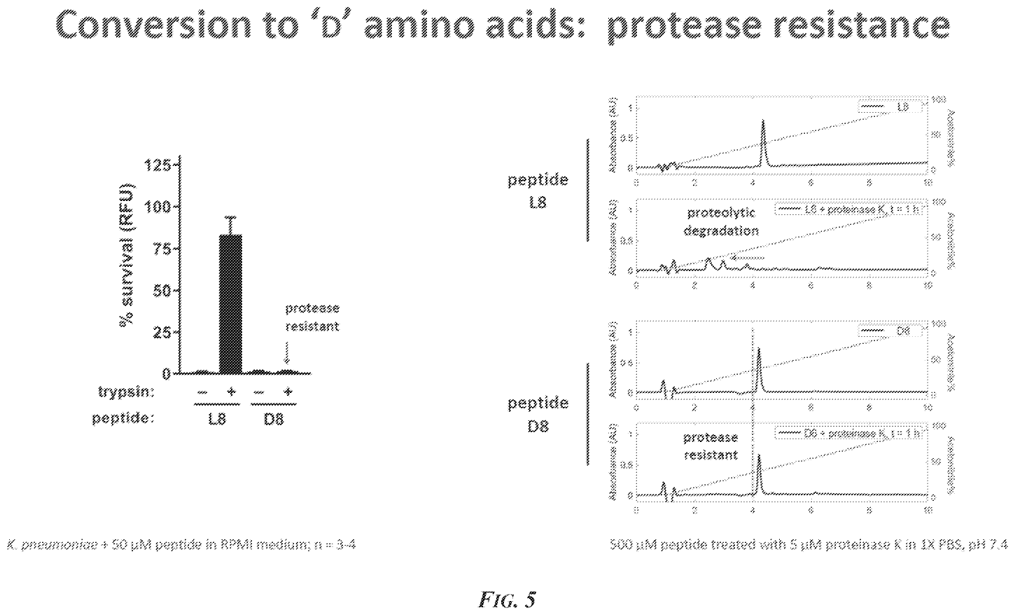

Generally, peptides that include L-amino acids are highly susceptible to proteolytic degradation and have other drawbacks like potential immunogenicity and, for CXCL10-derived peptides, host-targeted effects like causing chemotaxis and inflammation. Therefore, an all-D-amino acid-containing derivative of SEQ ID NO: 19 referred to as Peptide D8 was synthesized and tested for protease resistance. The results are shown in FIG. 5.

As shown in FIG. 5, Peptide L8 (SEQ ID NO: 19 with all L-amino acids) was degraded by proteases (see FIG. 5, right panel), resulting in a loss of activity (see FIG. 5, left panel). Peptide D8 (SEQ ID NO: 19 with all D-amino acids), on the other hand, was resistant to protease degradation and maintained bactericidal activity even after pretreatment with a protease (see FIG. 5, left panel). As a result, it was determined that a derivative of the 8-mer N-terminal antimicrobial domain of CXCL10 containing all D-amino acids was resistant to proteases and retained antimicrobial activity.

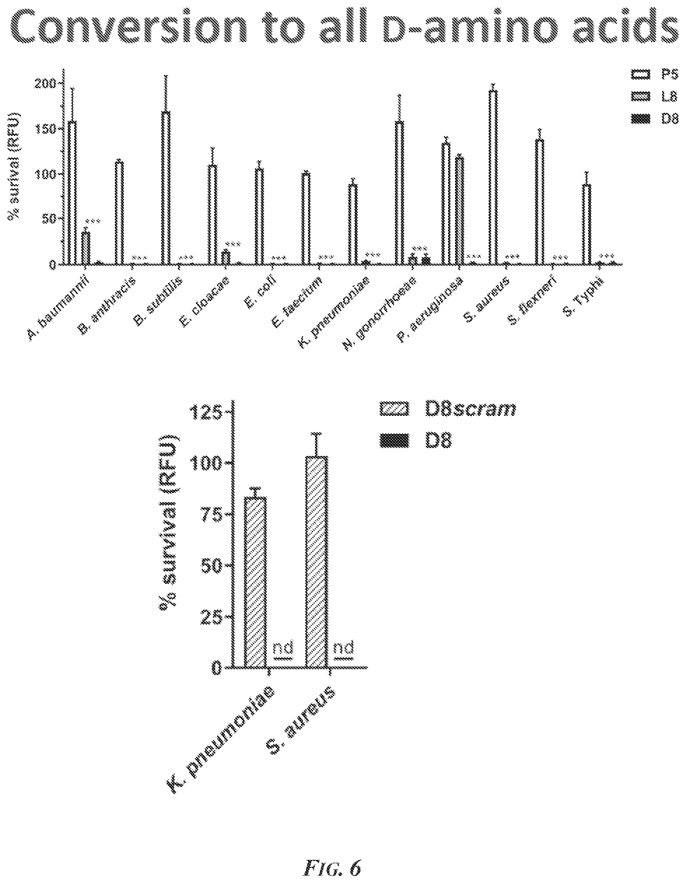

The antimicrobial activities of Peptide D8 (SEQ ID NO: 19 with all D-amino acids) against various bacteria was also tested using the alamarBlue assay. The bacteria tested were the same as those tested for Peptide P1 (SEQ ID NO: 2) in FIG. 2. The results are shown in FIG. 6.

As shown in FIG. 6, Peptide D8 (SEQ ID NO. 19 with all D-amino acids) displayed broad spectrum bactericidal activity against Gram-positive and Gram-negative organisms including Acinetobacter baumannii, Bacillus anthracis, Bacillus subtilis, Enterobacter cloacae, Escherichia coli, Enterococcus faecium, Klebsiella pneumoniae, Neisseria gonorrhoeae, Staphylococcus aureus, and Salmonella enterica serovar Typhi. Additionally, and unlike Peptide L8 (SEQ ID NO: 19 with all L-amino acids), Peptide D8 (SEQ ID NO: 19 with all D-amino acids) also displayed antibacterial activity against Pseudomonas aeruginosa.

### Example 4

Anti-K. pneumoniae Activity in a Wound Infection Model

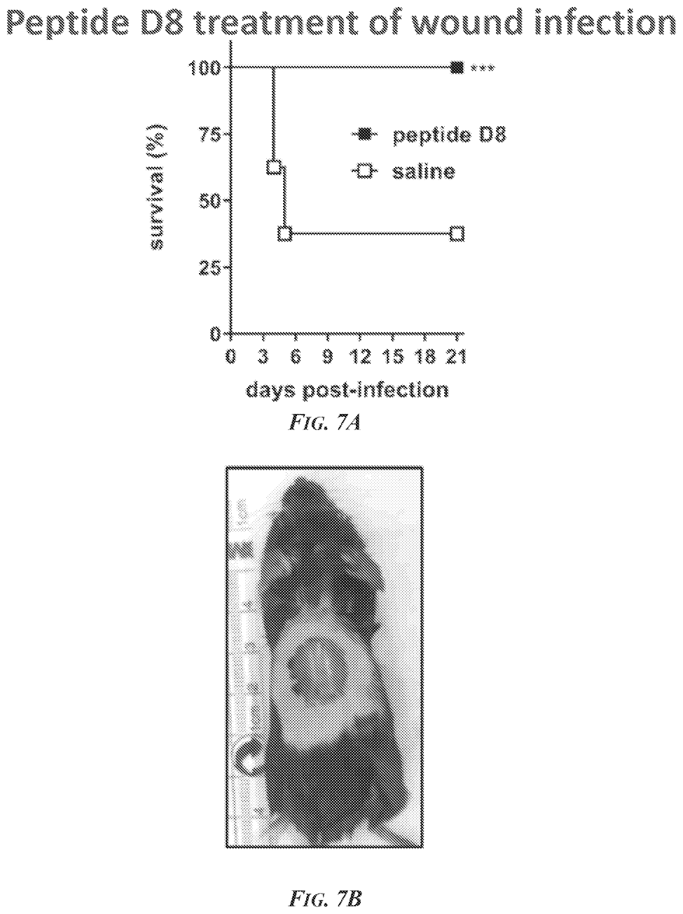

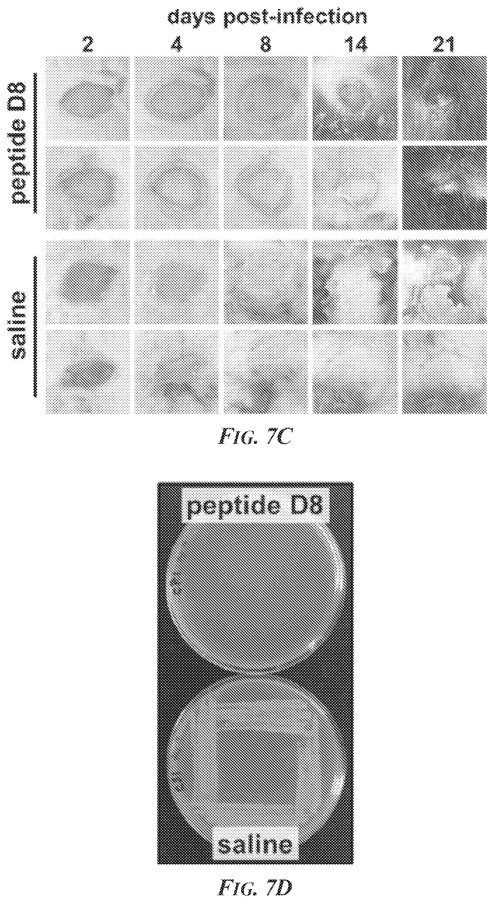

Whether Peptide D8 (SEQ ID NO: 19 with all D-amino acids) could prevent and/or cure wound infections, a murine model of surgical-site infection caused by K. pneumoniae was employed. Full-thickness wounds were generated in C57BL/6 mice and inoculated with an LD₅₀ (1×10³ cfu total) of K. pneumoniae ATCC 43816. Infected wounds were treated with 10 μl of 1.2% Peptide D8 (SEQ ID NO: 19 with all D-amino acids) prepared in saline, or an equivalent volume of saline alone, 4 hours post-infection, and then twice per day for 4 days. The results are shown in FIGS. 7A-7C.

As shown in FIGS. 7A and 7B, Peptide D8 (SEQ ID NO: 19 with all D-amino acids) completely prevented mortality over 21 days (FIG. 7A) and supported the resolution of infection and wound closure/healing (FIGS. 7B and 7C).

The growth of bacteria from swabs recovered from the wounds of animals treated with Peptide D8 (SEQ ID NO: 19 with all D-amino acids) or saline alone was also tested. As shown in FIG. 7D, whereas viable bacteria were cultured from wounds of animals treated with saline, no such viable bacteria were found in wounds of animals treated with Peptide D8 (SEQ ID NO: 19 with all D-amino acids).

### Example 5

Anti-K. pneumoniae Activities of Various Substitution Derivatives

Towards identifying the important molecular determinants of Peptide D8 (SEQ ID NO: 19 with all D-amino acids) and potential positions that are not essential to antimicrobial activity (and therefore can be modified to improve activity), an alanine scan was performed and the effects of individual substitution variants against K. pneumoniae were determined using alamarBlue. The results are shown in FIG. 8.

As shown in FIG. 8, the threonine residues at positions 2 and 6 of Peptide D8 (SEQ ID NO: 19 with all D-amino acids) could be substituted without affecting antimicrobial activity.

Since position 2 could be substituted, derivatives were produced with all other amino acids substituted into position 2. The alamarBlue assay was employed as a primary screen to identify those peptides that had significantly diminished activity against K. pneumoniae. Peptides that did well in the alamarBlue assay progressed to a secondary screen that employed more discriminating MIC methodology. The results are presented in FIG. 9A.

As shown in FIG. 9A, peptides with several amino acid two substitutions (including but not limited to tyrosine) showed strong antimicrobial activity. FIGS. 9B and 9C are photographs of microplates inoculated with K. pneumoniae in Mueller-Hinton II broth for minimum inhibitory concentration (MIC) determination over the indicated peptide concentrations (n=3-4). Underlined substitution variants outperformed the parent peptide D8.

Similarly, since position 6 could be substituted, derivatives were also produced in which all other amino acids were substituted into position 6. The alamarBlue assay was again employed as a primary screen to identify those peptides that had significantly diminished activity against K. pneumoniae. Peptides that did well in the alamarBlue assay progressed to a secondary screen that employed more discriminating MIC methodology. The results are presented in FIG. 10A.

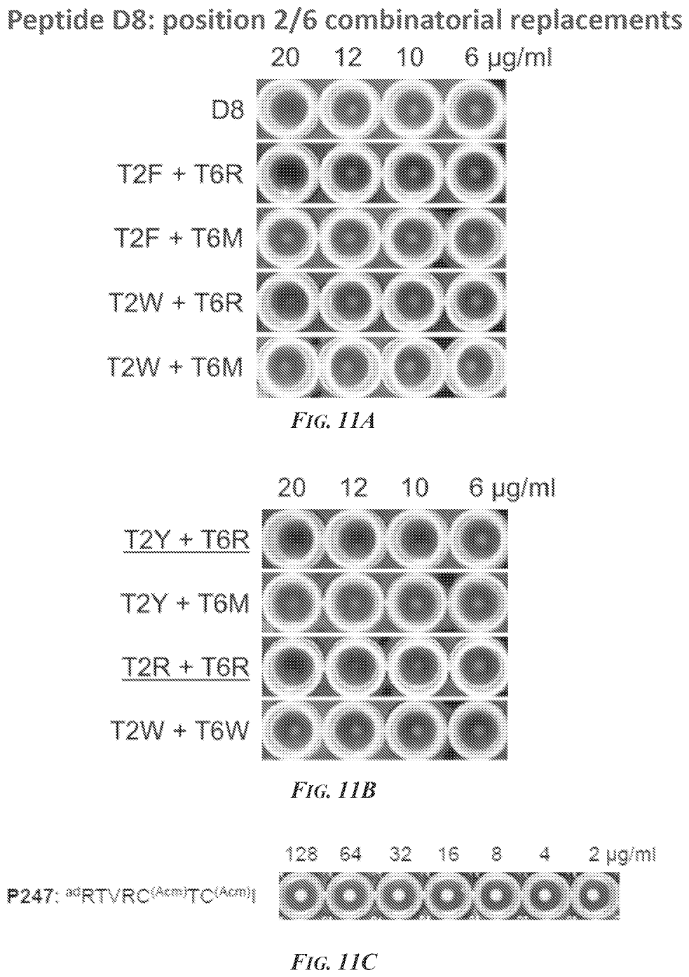

As shown in FIG. 10A, peptides with several amino acid 6 substitutions (including but not limited to arginine) showed strong antimicrobial activity. FIGS. 10B and 10C are photographs of microplates inoculated with K. pneumoniae in Mueller-Hinton II broth for minimum inhibitory concentration (MIC) determination over the indicated peptide concentrations (n=3-4). Underlined substitution variants outperformed the parent Peptide D8. Finally, combinations of substitutions at both positions 2 and 6 were also tested. The results are shown in FIGS. 11A and 11B. Using MIC determination, it was found that some combinations improved activity, but most did not.

From the D8 alanine scan, it was determined that the cysteines at positions 5 and 7 were essential to activity, so what effect capping the thiol groups of the cysteine residues would have was also tested. Asa shown in FIG. 11C, capping the cysteines abolished activity.

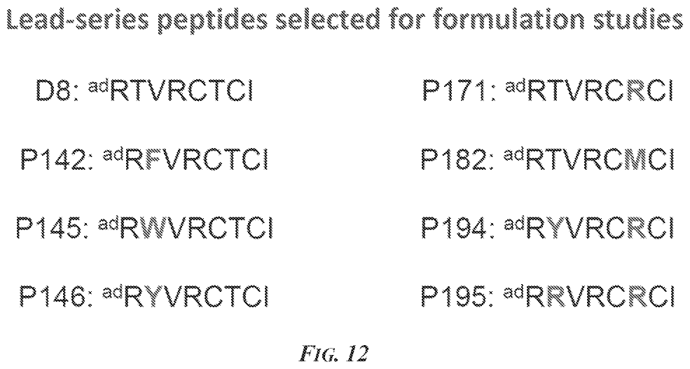

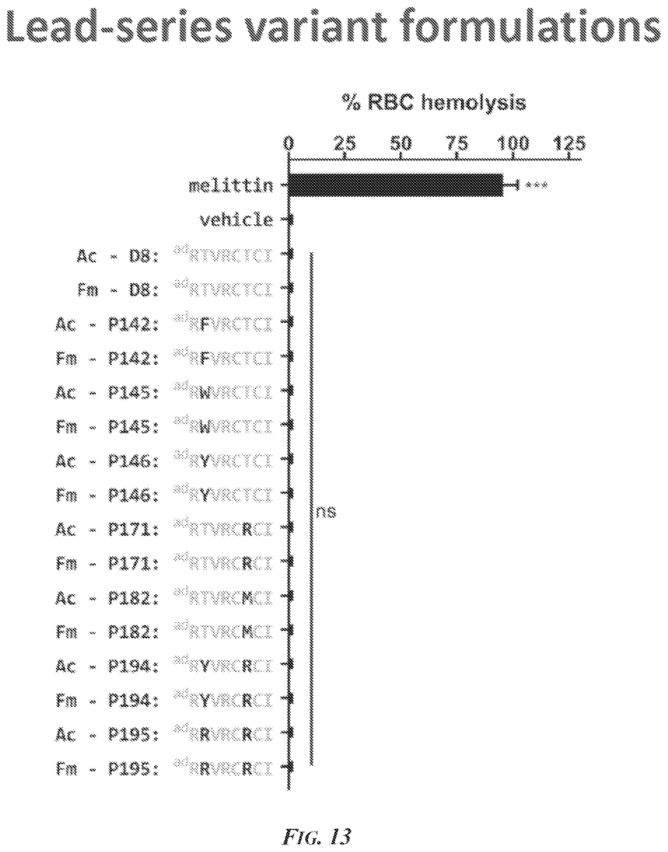

A summary of various peptides that demonstrated antimicrobial activity that was commensurate or improved compared to the parent Peptide D8 is shown in FIG. 12.

### Example 6

### Comparisons of Different Counterion Salts on Peptide Activities

Up to this point, the peptides tested were formulated as salts with trifluoroacetic acid (TFA) being the counterion. TFA, however, can be toxic, so a more biocompatible formulation was sought. Since the counterion can influence activity and peptide properties (including but not limited to solubility), two different possible formulations with respect to were tested with respect to inducing red blood cell hemolysis: acetate and formate. The results are summarized in FIG. 13.

As shown in FIG. 13, none of the various lead-series Peptides shown in FIG. 12 formulated as either an acetate (Ac) or formate (Fm) salt caused the lysis of human red blood cells.

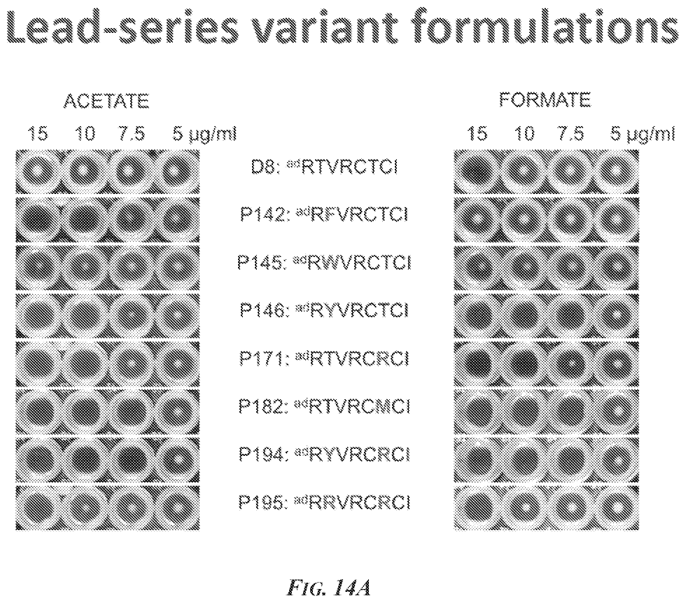

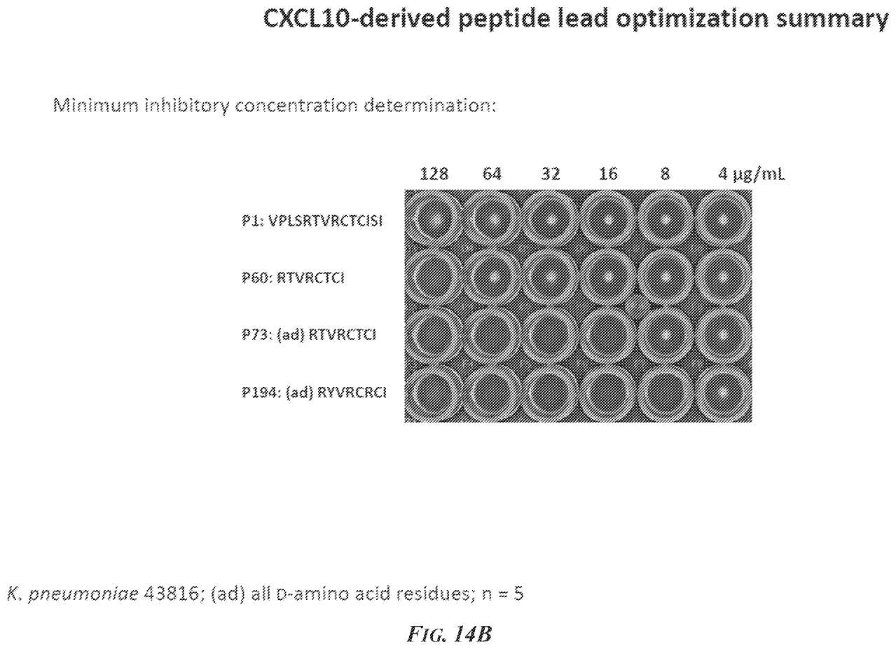

The antimicrobial effects of acetate and formate formulations of the lead-series Peptides were also tested using MIC determination. The results of minimum inhibitory concentration (MIC) determinations over the indicated peptide concentrations for K. pneumoniae in Mueller-Hinton II broth (n=3-4) are shown in FIGS. 14A and 14B. The activities of lead-series Peptide variants, produced as acetate or formate formulations, were compared, and the results are shown in FIG. 14A. FIG. 14B summarizes lead optimization using acetate formulations, from discovery of the original peptide (P1; SEQ ID NO: 1), through determining the minimal active sequence (P60; SEQ ID NO: 19), conversion to all D-amino acids (P73; SEQ ID NO: 19 with all D-amino acids), to the generation of substitution variants (P194; SEQ ID NO: 70 with all D-amino acids). Additional MIC data can be found in Tables 6-8.

Summarily, it appeared that the acetate salts had better activity profiles.

### Example 7

### Comparisons of Different Labeled Derivatives on Peptide Activities

To study peptide-mediated effects, it would be advantageous to label the peptides of the presently disclosed subject matter with various labels (e.g., detectable labels). Either alamarBlue or MIC determination was employed to test whether specific labels disrupted the activity of peptide derivatives based on the amino acid sequence of Peptide D8 (SEQ ID NO: 19 with all D-amino acids). Various modified peptides (i.e., derivatives) were created, including derivatives that were labeled with biotin, propargylglycine (PRA), including L-PRA and D-PRA derivatives, Phe(4-N₃), etc. (see Table 3).

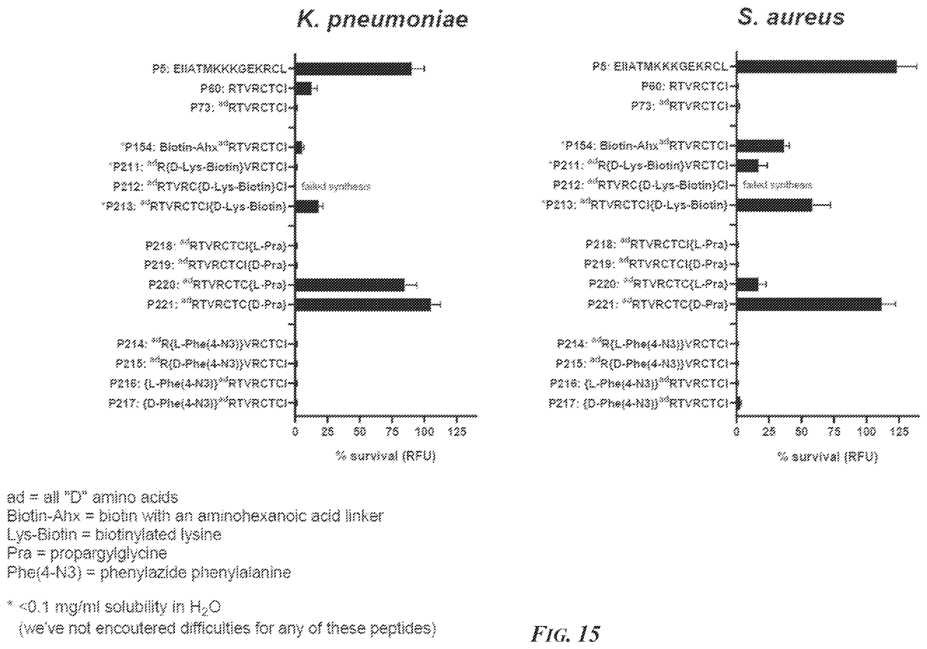

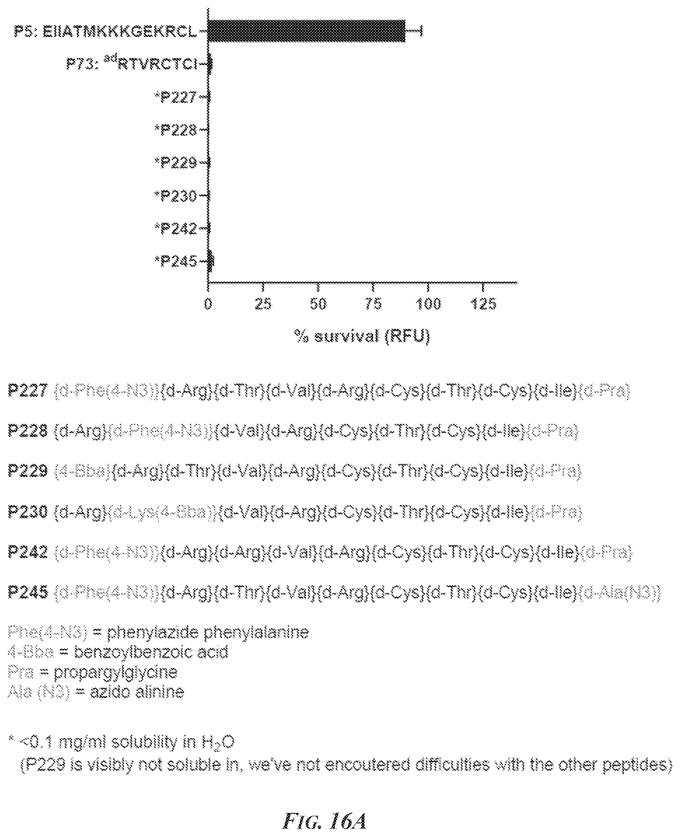

As set forth in FIG. 15, MDR K. pneumoniae BL13802 or methicillin-resistant S. aureus LAC were treated with 50 μM of the indicated singly modified peptides, composed primarily of D-amino acids, in RPMI medium for 2 hours at 37° C. is shown. Survival following peptide exposure was measured using the fluorescent viability reagent alamarBlue.

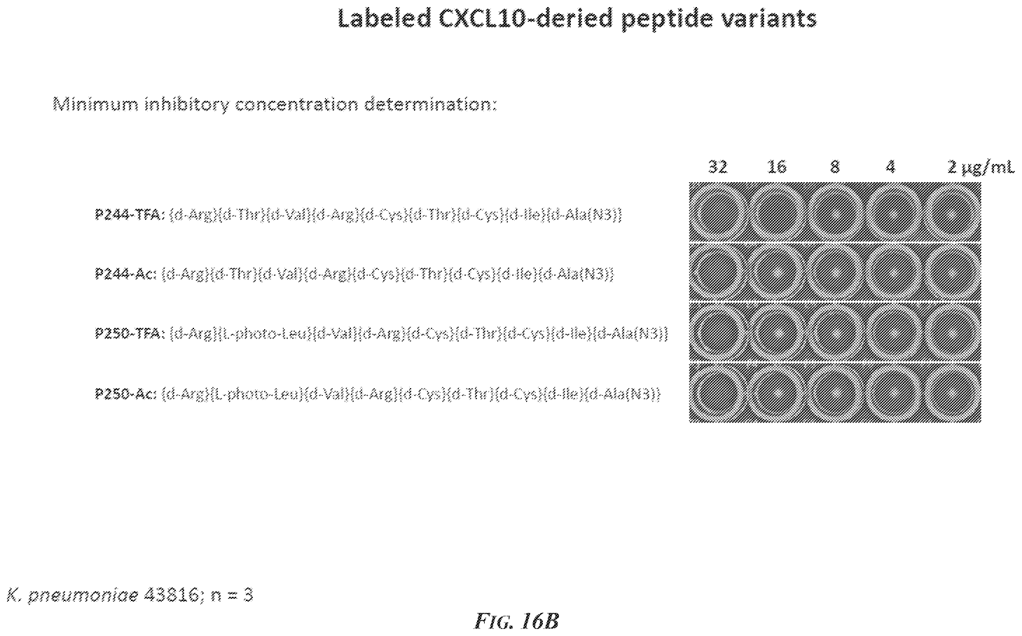

In FIGS. 16A and 16B, survival of MDR K. pneumoniae BL13802 treated with 50 μM of the indicated doubly modified peptides, composed primarily of D-amino acids, in RPMI medium for 2 hours at 37° C., is shown. Survival following peptide exposure was measured using the fluorescent viability reagent alamarBlue.

From the results presented in FIGS. 15, 16A, and 16B, it was apparent that some labels destroyed activity, but others had little or no effect. Those that were tolerated by Peptide D8 (SEQ ID NO: 19 with all D-amino acids) included photoaffinity labels and modifications that would allow “click chemistry. Additionally, the specific counterion had some impact on experimental outcomes.

### REFERENCES

All references listed below and/or in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990) 215 J Mol Biol 403-410.
Ausubel et al. (2002) Short Protocols in Molecular Biology, Fifth ed. John Wylie & Sons, New York, New York, United States of America.
Ausubel et al. (2003) Current Protocols in Molecular Biology, John Wylie & Sons, Inc, New York, New York, United States of America.
Bird et al. (1988) Science 242:423-426.
Bowie et al. (1990) Science 247:1306-1310.
Chou & Fasman (1974) Biochemistry 13:222-245.
Chou & Fasman (1978) Ann Rev Biochem 47: 251-276.
Chou & Fasman (1979) Biophys J 26:367-384.
Crawford et al. (2023) 9 ACS Infect Dis 122-139.
Dayhoff et al. (in Atlas of Protein Sequence and Structure 1978, National Biomedical Research Foundation, Washington D.C., United States of America.
Goodman el al. (1981) Perspectives in Peptide Chemistry pages 283-294.
Harlow & Lane (1988) Antibodies: A Laboratory Manual, Cold Spring Harbor Laboratory Press, Cold Spring Harbor, New York, United States of America.
Harlow & Lane (1999) Using Antibodies: A Laboratory Manual, Cold Spring Harbor Laboratory Press, Cold Spring Harbor, New York, United States of America.
Henikoff& Henikoff (1992) 89 Proc Natl Acad Sci USA 10915-10919.
Houston et al. (1988) Proc Natl Acad Sci USA 85:5879-5883.
Johnson & Overington (1993) J Mol Biol 233:716-738.
Karlin & Altschul (1993) 90 Proc Natl Acad Sci USA 5873-5877.
Kyte & Doolittle (1982) J Mol Biol 157:105-132.
Needleman & Wunsch (1970) 48 J Mol Biol 443-453.
Nielsen et al. (1991) Science 254:1497-1500.
Pearson & Lipman (1988) Proc Natl Acad Sci USA 85:2444-2448.
Remington et al. (1975) Remington's Pharmaceutical Sciences, 15^{th} ed., Mack Pub. Co., Easton, Pennsylvania, United States of America.
Smith & Waterman (1981) 2 Adv Appl Math 482-489.
U.S. Patent Application Publication No. 2011/0218140.
U.S. Pat. Nos. 4,522,752; 4,554,101: 5,034,506.
Wiegand et al. (2008) 3 Nature Protocols 163-175.

It will be understood that various details of the presently disclosed subject matter can be changed without departing from the scope of the presently disclosed subject matter. Furthermore, the foregoing description is for the purpose of illustration only, and not for the purpose of limitation.

TABLE 4
Fold Change MIC₅₀ Values for ‘Alanine-scan’ Substitution
Variants of CXCL 10-derived Peptide D8-acetate

| | D8 | Peptide D8 amino acid substitutions | | | | | | | |
|---|---|---|---|---|---|---|---|---|---|
| bacterial species/group | MIC₅₀^{a} | MIC₅₀ fold change | | | | | | | |
| (# of isolates tested) | (μg/mL) | R1A | T2A | V3A | R4A | C5A | T6A | C7A | I8A |
| Enterococcus faecalis (2) | 16 | 2 | 2 | >4 | >4 | >4 | 1 | >4 | 4 |
| Enterococcus faecium (3) | 4 | 2 | 2 | 8 | 8 | >16 | 1 | 16 | 4 |
| Acinetobacter spp.^{b} (5) | 2 | 4 | 2 | 32 | >32 | >32 | 2 | >32 | 2 |
| Klebsiella pneumoniae (5) | 8 | 4 | 2 | >8 | >8 | >8 | 2 | >8 | 4 |
| Escherichia coli (5) | 4 | 4 | 2 | 16 | >16 | >16 | 1 | >16 | 4 |
| Enterobacter spp.^{c} (5) | 16 | 2 | 1 | >4 | >4 | >4 | 0.5 | >4 | 1 |
| Pseudomonas aeruginosa (5) | 32 | 2 | >2 | >2 | >2 | >2 | 2 | >2 | 2 |
| Streptococcus pneumoniae (5) | 64 | —^{d} | — | — | — | — | 1 | — | — |
| Staphylococcus aureus (5) | >64 | — | — | — | — | — | — | — | — |

^{a}Minimum inhibitory concentration that prevents the growth of 50% of the tested isolates.
^{b}A. baumannii-calcoaceticus complex.
^{c}E. cloacae complex.
^{d}undefined.

TABLE 5
Fold Change MIC₅₀ Values for ‘Alanine-scan’ Substitution
Variants of CXCL10-derived Peptide P194-acetate

| | P194 | Peptide P194 amino acid substitutions | | | | | | | |
|---|---|---|---|---|---|---|---|---|---|
| bacterial species/group | MIC₅₀^{a} | MIC₅₀ fold change | | | | | | | |
| (# of isolates tested) | (μg/mL) | R1A | T2A | V3A | R4A | C5A | T6A | C7A | I8A |
| Enterococcus faecalis (2) | 2 | 1 | 4 | 32 | 4 | >32 | 4 | 32 | 16 |
| Enterococcus faecium (3) | 1 | 2 | 2 | 8 | 4 | 64 | 4 | 16 | 4 |
| Acinetobacter spp.^{b} (5) | 4 | 1 | 0.5 | 4 | 2 | >16 | 4 | 4 | 1 |
| Klebsiella pneumoniae (5) | 4 | 1 | 2 | 4 | 4 | >16 | 8 | 16 | 8 |
| Escherichia coli (5) | 8 | 0.5 | 0.5 | 2 | 1 | >8 | 2 | 4 | 2 |
| Enterobacter spp.^{c} (5) | 16 | 0.5 | 0.5 | 4 | 1 | >4 | 2 | 2 | 1 |
| Pseudomonas aeruginosa (5) | 32 | 1 | >2 | >2 | >2 | >2 | >2 | >2 | >2 |
| Streptococcus pneumoniae (5) | 32 | 1 | 2 | >2 | 2 | >2 | 2 | >2 | >2 |
| Staphylococcus aureus (5) | 32 | 0.5 | 2 | >2 | >2 | >2 | >2 | >2 | >2 |

^{a}Minimum inhibitory concentration that prevents the growth of 50% of the tested isolates.
^{b}A. baumannii-calcoaceticus complex.
^{c}E. cloacae complex.

TABLE 6
Exemplary Peptides with SEQ ID NOs.,
Sequences, and Peptide Names

| SEQ | | |
|---|---|---|
| ID NO: | Sequence | Peptide Name |
| 19 | RTVRCTCI | D8 |
| 23 | RAVRCTCI | D8-T2A |
| 30 | RRVRCTCI | D8-T2R |
| 31 | RNVRCTCI | D8-T2N |
| 32 | RDVRCTCI | D8-T2D |
| 33 | RCVRCTCI | D8-T2C |
| 35 | RQVRCTCI | D8-T2Q |
| 34 | REVRCTCI | D8-T2E |
| 37 | RHVRCTCI | D8-T2H |
| 38 | RIVRCTCI | D8-T2I |
| 39 | RLVRCTCI | D8-T2L |
| 40 | RKVRCTCI | D8-T2K |
| 41 | RMVRCTCI | D8-T2M |
| 42 | REVRCTCI | D8-T2F |
| 43 | RPVRCTCI | D8-T2P |
| 36 | RGVRCTCI | D8-T2G |
| 44 | RSVRCTCI | D8-T2S |
| 45 | RWVRCTCI | D8-T2W |
| 46 | RYVRCTCI | D8-T2Y |
| 47 | RVVRCTCI | D8-T2V |
| 98 | R[alloThr]VRCTCI | D8-T2alloThr |
| 99 | R[MeThr]VRCTCI | D8-T2MeThr |
| 100 | R[Abu]VRCTCI | D8-T2Abu |
| 101 | R[NapthylAla]VRCTCI | D8-T2NapAla |
| 19 | RTVRCTCI | D8 |
| 27 | RTVRCACI | D8-T6A |
| 48 | RTVRCRCI | D8-T6R |
| 49 | RTVRCNCI | D8-T6N |
| 50 | RTVRCDCI | D8-T6D |
| 51 | RTVRCCCI | D8-T6C |
| 53 | RTVRCQCI | D8-T6Q |
| 52 | RTVRCECI | D8-T6E |
| 54 | RTVRCGCI | D8-T6G |
| 55 | RTVRCHCI | D8-T6H |
| 56 | RTVRCICI | D8-T6I |
| 57 | RTVRCLCI | D8-T6L |
| 58 | RTVRCKCI | D8-T6K |
| 59 | RTVRCMCI | D8-T6M |
| 60 | RTVRCFCI | D8-T6F |
| 61 | RTVRCPCI | D8-T6P |
| 62 | RTVRCSCI | D8-T6S |
| 63 | RTVRCWCI | D8-T6W |
| 64 | RTVRCYCI | D8-T6Y |
| 65 | RTVRCVCI | D8-T6V |
| 102 | RTVRC[alloThr]CI | D8-T6alloThr |
| 103 | RTVRC[MeThr]CI | D8-T6MeThr |
| 104 | RTVRC[Abu]CI | D8-T6Abu |
| 105 | RTVRC[NapthylAla ]CI | D8-T6NapAla |
| 19* | PEG2-RTVRCTCI | PEG2-D8 |
| 19* | PEG4-RTVRCTCI | PEG4-D8 |
| 70 | RYVRCRCI | P194 |
| 106 | AYVRCRCI | P194-R1A |
| 107 | NYVRCRCI | P194-R1N |
| 108 | DYVRCRCI | P194-R1D |
| 109 | CYVRCRCI | P194-R1C |
| 110 | QYVRCRCI | P194-R1Q |
| 111 | EYVRCRCI | P194-R1E |
| 112 | GYVRCRCI | P194-R1G |
| 113 | HYVRCRCI | P194-R1H |
| 114 | IYVRCRCI | P194-R1I |
| 115 | LYVRCRCI | P194-R1L |
| 116 | KYVRCRCI | P194-R1K |
| 117 | MYVRCRCI | P194-R1M |
| 118 | FYVRCRCI | P194-R1F |
| 119 | PYVRCRCI | P194-R1P |
| 120 | SYVRCRCI | P194-R1S |
| 121 | TYVRCRCI | P194-R1T |
| 122 | WYVRCRCI | P194-R1W |
| 123 | YYVRCRCI | P194-R1Y |
| 124 | VYVRCRCI | P194-R1V |
| 125 | [Cit]YVRCRCI | P194-R1Cit |
| 126 | [BR]YVRCRCI | P194-R1hR |
| 127 | [R(NO₂)]YVRCRCI | P194-R1R(NO2) |
| 128 | [Agb]YVRCRCI | P194-R1Agb |
| 70 | RYVRCRCI | P194 |
| 129 | RAVRCRCI | P194-Y2A |
| 130 | RRVRCRCI | P194-Y2R |
| 131 | RNVRCRCI | P194-Y2N |
| 132 | RDVRCRCI | P194-Y2D |
| 133 | RCVRCRCI | P194-Y2C |
| 134 | RQVRCRCI | P194-Y2Q |
| 135 | REVRCRCI | P194-Y2E |
| 136 | RGVRCRCI | P194-Y2G |
| 137 | RHVRCRCI | P194-Y2H |
| 138 | RIVRCRCI | P194-Y2I |
| 139 | RLVRCRCI | P194-Y2L |
| 140 | RKVRCRCI | P194-Y2K |
| 141 | RMVRCRCI | P194-Y2M |
| 142 | RFVRCRCI | P194-Y2F |
| 143 | RPVRCRCI | P194-Y2P |
| 144 | RSVRCRCI | P194-Y2S |
| 145 | RTVRCRCI | P194-Y2T |
| 146 | RWVRCRCI | P194-Y2W |
| 147 | RVVRCRCI | P194-Y2V |
| 148 | R[MeTyr]VRCRCI | P194-Y2MeTyr |
| 149 | R[Iodo-Tyr]VRCRCI | P194-Y2hIodoTyr |
| 150 | R[DimethylTyr]VRCRCI | P194-Y2DiMethylTyr |
| 151 | R[hP]VRCRCI | P194-Y2hP |
| 70* | PEG2-RYVRCRCI | PEG2-P194 |
| 70* | PEG4-RYVRCRCI | PEG4-P194 |

alloThr: allothreonine;
MeThr: o-methyl threonine;
Abu: aminobutyric;
NapthylAla: ala(2naphthyl);
MeThr: allothreonine;
PEG2: two PEG groups;
PEG4: 4 PEG groups;
Cit: citrulline;
hR: homoarginine;
R(NO₂): nitroarginine;
Agb: norarginine;
MeTyr: methyl tyrosine;
Iodo-Tyr: 3-iodo tyrosine;
DimethylTyr: dimethyl tyrosine;
hP: homoproline

TABLE 7
MIC Values of Various Peptide Formulations Against Representative Bacteria

| | | | MIC (mg/L) | | | | | | |
|---|---|---|---|---|---|---|---|---|---|
| Collection | | | D8 | D8 | P182 | P182 | P194 | P194 | |
| No. | Organism* | Category | A¹ | F¹ | A¹ | F¹ | A¹ | F¹ | Levofloxacin |
| 1204424 | ABC species complex | non-MDR | 4 | 8 | 16 | 16 | 8 | 16 | 0.12 |
| 1208060 | ABC species complex | non-MDR | 4 | 8 | 16 | 16 | 8 | 16 | 0.06 |
| 1215088 | ABC species complex | MDR | 4 | 8 | 16 | 16 | 8 | 16 | 32 |
| 1215098 | ABC species complex | MDR | 8 | 8 | 16 | 16 | 16 | 32 | 16 |
| 1216520 | ABC species complex | non-MDR | 4 | 8 | 16 | 16 | 8 | 16 | 0.25 |
| 1230117 | Enterobacter cloacae | non-MDR | 16 | 32 | 16 | 16 | 8 | 16 | 0.015 |
| 1197959 | E. cloacae species complex | non-MDR | 16 | 32 | 32 | 32 | 16 | 16 | 0.25 |
| 1204419 | B. cloacae species complex | non-MDR | 16 | 16 | 16 | 16 | 16 | 16 | 0.03 |
| 1230601 | E. cloacae species complex | non-MDR | 16 | 16 | 16 | 32 | 16 | 16 | 0.03 |
| 1232701 | E. cloacae species complex | MDR | 16 | 32 | 32 | 32 | 16 | 32 | 8 |
| 1204777 | Enterococcus faecalis | VRE | 16 | 16 | 8 | 8 | 4 | 8 | 32 |
| 1211589 | Enterococcus faecalis | VRE | 16 | 32 | 8 | 8 | 4 | 8 | 32 |
| ATCC 29212 | Enterococcus faecalis | QC | 16 | 32 | 8 | 8 | 4 | 8 | 0.5 |
| ATCC 29212 | Enterococcus faecalis | QC | 16 | 32 | 8 | 8 | 4 | 8 | 0.5 |
| ATCC 29212 | Enterococcus faecalis | QC | 16 | 32 | 8 | 8 | 4 | 8 | 0.5 |
| 1196024 | Enterococcus faecium | VRE | 4 | 8 | 8 | 8 | 2 | 4 | >64 |
| 1208412 | Enterococcus faecium | VRE | 4 | 8 | 8 | 8 | 2 | 4 | >64 |
| 1222807 | Enterococcus faecium | VSE | 4 | 8 | 8 | 8 | 2 | 4 | 8 |
| 1191008 | Escherichia coli | MDR, ESBL | 8 | 8 | 16 | 8 | 8 | 8 | 32 |
| 1231795 | Escherichia coli | MDR, ESBL | 8 | 16 | 16 | 16 | 8 | 16 | 8 |
| 1232428 | Escherichia coli | MDR (COL-R), ESBL | 8 | 16 | 16 | 16 | 16 | 16 | 8 |
| 1248389 | Escherichia coli | ESBL | 8 | 8 | 16 | 16 | 8 | 8 | 8 |
| ATCC 25922 | Escherichia coli | QC | 8 | 8 | 16 | 16 | 8 | 16 | 0.015 |
| ATCC 25922 | Escherichia coli | QC | 8 | 16 | 16 | 16 | 4 | 16 | 0.015 |
| ATCC 25922 | Escherichia coli | QC | 8 | 8 | 16 | 16 | 8 | 16 | 0.015 |
| ATCC 25922 | Escherichia coli | QC | 8 | 16 | 16 | 16 | 8 | 16 | 0.015 |
| ATCC BAA-2452 | Escherichia coli | NDM-1 | 4 | 8 | 16 | 16 | 8 | 16 | 0.03 |
| 1230887 | Klebsiella pneumoniae | non-MDR | >64 | >64 | 64 | 64 | 64 | >64 | 0.06 |
| 1236064 | Klebsiella pneumoniae | non-MDR | 16 | 16 | 16 | 32 | 8 | 16 | 0.03 |
| ATCC 43816 | Klebsiella pneumoniae | sponsor | 8 | 16 | 16 | 16 | 8 | 8 | 0.12 |
| ATCC 43816 | Klebsiella pneumoniae | sponsor | 8 | 16 | 16 | 16 | 8 | 8 | 0.03 |
| ATCC 43816 | Klebsiella pneumoniae | sponsor | 16 | 32 | 32 | 32 | 8 | 16 | 0.06 |
| ATCC 43816 | Klebsiella pneumoniae | sponsor | 16 | 16 | 16 | 16 | 4 | 8 | 0.03 |
| ATCC 700603 | Klebsiella pneumoniae | ESBL | 8 | 16 | 32 | 32 | 8 | 16 | 1 |
| ATCC BAA 1705 | Klebsiella pneumoniae | KPC | 16 | 32 | 16 | 32 | 8 | 16 | 64 |
| 1190985 | Pseudomonas aeruginosa | non-MDR | 32 | 64 | 64 | 64 | 32 | 64 | 0.25 |
| 1202638 | Pseudomonas aeruginosa | MDR | 32 | 64 | 64 | 64 | 32 | 64 | 16 |
| 1210081 | Pseudomonas aeruginosa | non-MDR | 32 | 64 | 64 | 64 | 32 | 64 | 0.5 |
| 1242554 | Pseudomonas aeruginosa | non-MDR | 16 | 32 | 32 | 32 | 32 | 32 | 0.5 |
| 1263496 | Pseudomonas aeruginosa | MDR | 16 | 16 | 16 | 16 | 16 | 16 | 0.25 |
| ATCC 27853 | Pseudomonas aeruginosa | QC | 16 | 16 | 32 | 16 | 16 | 16 | 1 |
| ATCC 27853 | Pseudomonas aeruginosa | QC | 16 | 16 | 16 | 16 | 16 | 16 | 0.5 |
| ATCC 27853 | Pseudomonas aeruginosa | QC | 16 | 16 | 16 | 16 | 16 | 16 | 1 |
| 1124691 | Staphylococcus aureus | MRSA (USA300)/MDR | >64 | >64 | 64 | 32 | 32 | 32 | 8 |
| 1189667 | Staphylococcus aureus | MRSA/MDR | 64 | >64 | 16 | 16 | 32 | 32 | >64 |
| 1189809 | Staphylococcus aureus | MRSA/MDR | >64 | >64 | 32 | 32 | 32 | 64 | 32 |
| 1190388 | Staphylococcus aureus | MRSA | >64 | >64 | 32 | 32 | 32 | 32 | 16 |
| 1221979 | Staphylococcus aureus | MRSA | >64 | >64 | 64 | 64 | 32 | 32 | 1 |
| ATCC 29213 | Staphylococcus aureus | QC | >64 | >64 | 32 | 32 | 32 | 32 | 0.12 |
| ATCC 29213 | Staphylococcus aureus | QC | >64 | >64 | 32 | 32 | 32 | 32 | 0.12 |
| ATCC 29213 | Staphylococcus aureus | QC | >64 | >64 | 32 | 32 | 32 | 32 | 0.25 |
| 1200385 | Streptococcus pneumoniae | PEN-S | 64 | >64 | 32 | 32 | 32 | 32 | 1 |
| 1219456 | Streptococcus pneumoniae | PEN-R | 64 | >64 | 32 | 32 | 32 | 32 | 1 |
| 1219876 | Streptococcus pneumoniae | PEN-R | 32 | >64 | 32 | 32 | 32 | 32 | 1 |
| 1223661 | Streptococcus pneumoniae | PEN-R | 64 | >64 | 32 | 32 | 32 | 32 | 1 |
| 1227224 | Streptococcus pneumoniae | PEN-S | 64 | 64 | 32 | 32 | 16 | 32 | 1 |
| ATCC 49619 | Streptococcus pneumoniae | QC | 64 | >64 | 32 | 32 | 16 | 32 | 0.5 |
| ATCC 49619 | Streptococcus pneumoniae | QC | 32 | >64 | 32 | 32 | 16 | 32 | 0.5 |
| ATCC 49619 | Streptococcus pneumoniae | QC | 64 | >64 | 32 | 32 | 16 | 32 | 0.5 |

*ABC: Acinetobacter baumannii-calcoaceticus
¹A: Acetate; F: formate; D8: SEQ ID NO: 19 with all D amino acids; P182: SEQ ID NO: 59; P194: SEQ ID NO: 70.

TABLE 8
MIC Values for Acetate Formulations of Peptides D8 (SEQ ID NO: 19) and P194 (SEQ ID NO: 70)

| | | | R1 | R2 | R1 | R2 |
|---|---|---|---|---|---|---|
| Collection No | Organism | Category | D8 | D8 | P194 | P194 |
| 1204424 | Acinetobacter baumannii-calcoaceticus species complex | non-MDR | 4 | 2 | 8 | 8 |
| 1208060 | Acinetobacter baumannii-calcoaceticus species complex | non-MDR | 4 | 2 | 8 | 4 |
| 1215088 | Acinetobacter baumannii-calcoaceticus species complex | MDR | 4 | 2 | 8 | 4 |
| 1215098 | Acinetobacter baumannii-calcoaceticus species complex | MDR | 8 | 4 | 16 | 8 |
| 1216520 | Acinetobacter baumannii-calcoaceticus species complex | non-MDR | 4 | 2 | 8 | 4 |
| 1230117 | Enterobacter cloacae | non-MDR | 16 | 16 | 8 | 8 |
| 1197959 | Enterobacter cloacae species complex | non-MDR | 16 | 16 | 16 | 16 |
| 1204419 | Enterobacter cloacae species complex | non-MDR | 16 | 8 | 16 | 16 |
| 1230601 | Enterobacter cloacae species complex | non-MDR | 16 | 8 | 16 | 8 |
| 1232701 | Enterobacter cloacae species complex | MDR | 16 | 16 | 16 | 16 |
| 1204777 | Enterococcus faecalis | VRE | 16 | 16 | 4 | 2 |
| 1211589 | Enterococcus faecalis | VRE | 16 | 16 | 4 | 2 |
| ATCC 29212 | Enterococcus faecalis | QC | 16 | 16 | 4 | 4 |
| ATCC 29212 | Enterococcus faecalis | QC | 16 | 16 | 4 | 4 |
| ATCC 29212 | Enterococcus faecalis | QC | 16 | 16 | 4 | 4 |
| 1196024 | Enterococcus faecium | VRE | 4 | 4 | 2 | 2 |
| 1208412 | Enterococcus faecium | VRE | 4 | 4 | 2 | 1 |
| 1222807 | Enterococcus faecium | VSE | 4 | 2 | 2 | 1 |
| 1191008 | Escherichia coli | MDR, ESBL | 8 | 4 | 8 | 8 |
| 1231795 | Escherichia coli | MDR, ESBL | 8 | 4 | 8 | 8 |
| 1232428 | Escherichia coli | MDR (COL-R), ESBL | 8 | 4 | 16 | 8 |
| 1248389 | Escherichia coli | ESBL | 8 | 4 | 8 | 8 |
| ATCC 25922 | Escherichia coli | QC | 8 | 4 | 8 | 4 |
| ATCC 25922 | Escherichia coli | QC | 8 | 4 | 4 | 8 |
| ATCC 25922 | Escherichia coli | QC | 8 | 4 | 8 | 8 |
| ATCC 25922 | Escherichia coli | QC | 8 | | 8 | |
| ATCC BAA-2452 | Escherichia coli | NDM-1 | 4 | 4 | 8 | 4 |
| 1230887 | Klebsiella pneumoniae | non-MDR | >64 | 64 | 64 | 64 |
| 1236064 | Klebsiella pneumoniae | non-MDR | 16 | 8 | 8 | 4 |
| ATCC 43816 | Klebsiella pneumoniae | sponsor | 8 | 8 | 8 | 8 |
| ATCC 43816 | Klebsiella pneumoniae | sponsor | 8 | 8 | 8 | 4 |
| ATCC 43816 | Klebsiella pneumoniae | sponsor | 16 | 8 | 8 | 4 |
| ATCC 43816 | Klebsiella pneumoniae | sponsor | 16 | — | 4 | — |
| ATCC 700603 | Klebsiella pneumoniae | ESBL | 8 | 4 | 8 | 8 |
| ATCC BAA 1705 | Klebsiella pneumoniae | KPC | 16 | 8 | 8 | 4 |
| 1190985 | Pseudomonas aeruginosa | non-MDR | 32 | 32 | 32 | 32 |
| 1202638 | Pseudomonas aeruginosa | MDR | 32 | 32 | 32 | 32 |
| 1210081 | Pseudomonas aeruginosa | non-MDR | 32 | 32 | 32 | 32 |
| 1242554 | Pseudomonas aeruginosa | non-MDR | 16 | 16 | 32 | 16 |
| 1263496 | Pseudomonas aeruginosa | MDR | 16 | 16 | 16 | 8 |
| ATCC 27853 | Pseudomonas aeruginosa | QC | 16 | 8 | 16 | 8 |
| ATCC 27853 | Pseudomonas aeruginosa | QC | 16 | 8 | 16 | 8 |
| ATCC 27853 | Pseudomonas aeruginosa | QC | 16 | 8 | 16 | 8 |
| 1124691 | Staphylococcus aureus | MRSA (USA300)/MDR | >64 | — | 32 | — |
| 1182822 | Staphylococcus aureus | | — | >64 | — | 32 |
| 1189667 | Staphylococcus aureus | MRSA/MDR | 64 | 64 | 32 | 16 |
| 1189809 | Staphylococcus aureus | MRSA/MDR | >64 | >64 | 32 | 32 |
| 1190388 | Staphylococcus aureus | MRSA | >64 | >64 | 32 | 16 |
| 1221979 | Staphylococcus aureus | MRSA | >64 | >64 | 32 | 32 |
| ATCC 29213 | Staphylococcus aureus | QC | >64 | 64 | 32 | 32 |
| ATCC 29213 | Staphylococcus aureus | QC | >64 | 64 | 32 | 32 |
| ATCC 29213 | Staphylococcus aureus | QC | >64 | 64 | 32 | 32 |
| 1200385 | Streptococcus pneumoniae | PEN-S | 64 | 64 | 32 | 32 |
| 1219456 | Streptococcus pneumoniae | PEN-R | 64 | 64 | 32 | 32 |
| 1219876 | Streptococcus pneumoniae | PEN-R | 32 | 64 | 32 | 32 |
| 1223661 | Streptococcus pneumoniae | PEN-R | 64 | 64 | 32 | 32 |
| 1227224 | Streptococcus pneumoniae | PEN-S | 64 | 64 | 16 | 32 |
| ATCC 49619 | Streptococcus pneumoniae | QC | 64 | 32 | 16 | 16 |
| ATCC 49619 | Streptococcus pneumoniae | QC | 32 | 64 | 16 | 16 |
| ATCC 49619 | Streptococcus pneumoniae | QC | 64 | 64 | 16 | 16 |

1. A peptide comprising, consisting essentially of, or consisting of the amino acid sequence RFVRCTCI (SEQ ID NO: 42), RWVRCTCI (SEQ ID NO: 45). RYVRCTCI (SEQ ID NO: 46), RTVRCRCI (SEQ ID NO: 48), RTVRCMCI (SEQ ID NO: 59), RYVRCRCI (SEQ ID NO: 70), RRVRCRCI (SEQ ID NO: 72), RWVRCWCI (SEQ ID NO: 73), a modified peptide thereof, or a fragment thereof, optionally wherein one or more of the amino acids of the peptide, further optionally all of the amino acids of the peptide, are D-amino acids.

2. The peptide of claim 1, wherein the peptide has bactericidal and/or bacteriostatic activity against a bacterium selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to Salmonella enterica serovars such as but not limited to Salmonella enterica serovar Typhi and Shigella flexneri; and biothreat agents such as but not limited to Bacillus anthracis in both vegetative and spore forms.

3. The peptide of claim 2, wherein the bacterium is an MDR strain of Enterococcus faecium, Staphylococcus aureus, Klebsiella pneumoniae, Acinetobacter baumannii, Pseudomonas aeruginosa, Salmonella enterica, optionally Salmonella enterica serovar Typhi, or Shigella flexneri.

4. The peptide of claim 1, wherein the peptide comprises, consists essentially of, or consists of the amino acid sequence RTVRCRCI (SEQ ID NO: 48), RTVRCMCI (SEQ ID NO: 59), or RYVRCRCI (SEQ ID NO: 70), a modified peptide thereof, or a fragment thereof.

5. A peptide comprising, consisting essentially of, or consisting of the amino acid sequence RTVRCRCI (SEQ ID NO: 48), RTVRCMCI (SEQ ID NO: 59), or RYVRCRCI (SEQ ID NO: 70), a modified peptide thereof, or a fragment thereof.

6. The peptide of claim 1, wherein one or more, optionally all, amino acids are D-amino acids.

7. The peptide of claim 1, wherein the peptide is polymer-functionalized, encapsulated in a particle, embedded in and/or on a solid support, optionally wherein the peptide is formulated for release from the solid support, impregnated in a dressing, optionally wherein the peptide is formulated for release from the dressing, and/or is formulated for use in a nebulizer, for topical administration, and/or for systemic administration.

8. The peptide of claim 1, wherein the peptide is a modified peptide that comprises a modification at the N-terminus, the C-terminus, or both the N-terminus and the C-terminus, optionally wherein the N-terminal and/or the C-terminal modification is selected from the group consisting of an addition or an aminohexanoic acid (AHX), an azido alanine (Ala(N₃)), a propargylglycine (PRA), a photoaffinity label, optionally a benzoylbenzoic acid (4-BBA), an azido phenylalanine (Phe(4-N₃)), a diazirine-containing amino acid, optionally leucine, methionine, lysine, proline, or phenylalanine; and/or is a replacement of a phenylalanine with an azido phenylalanine (Phe(4-N₃)), further optionally wherein the peptide that is modified is selected from the group consisting of SEQ ID NOs: 19, 20, 40, 42, 58, and 74-90.

9. A conjugate comprising a first peptide comprising, consisting essentially of, or consisting of the amino acid sequence RTVRCRCI (SEQ ID NO: 48), RTVRCMCI (SEQ ID NO: 59), or RYVRCRCI (SEQ ID NO: 70), a modified peptide thereof, or a fragment thereof, conjugated to a second peptide, optionally wherein the second peptide also comprises, consists essentially of, or consists of the amino acid sequence RTVRCRCI (SEQ ID NO: 48), RTVRCMCI (SEQ ID NO: 59), or RYVRCRCI (SEQ ID NO: 70).

10. The conjugate peptide of claim 9, wherein the first peptide is directly conjugated to the second peptide or the first and second peptides are indirectly conjugated to each other via a linker.

11. The conjugate of claim 10, wherein the linker is a peptide linker comprising 1-9 amino acids, optionally wherein the 1-9 amino acids are each individually selected from the group consisting of glycine and serine.

12. The conjugate of claim 9, wherein the conjugate is polymer-functionalized, encapsulated in a particle, embedded in and/or on a solid support, optionally wherein the peptide is formulated for release from the solid support, impregnated in a dressing, optionally wherein the peptide is formulated for release from the dressing, and/or is formulated for use in a nebulizer, for topical administration, and/or for systemic administration.

13. A conjugate comprising one or more peptides comprising, consisting essentially of, or consisting of the amino acid sequence RTVRCRCI (SEQ ID NO: 48), the amino acid sequence RTVRCMCI (SEQ ID NO: 59), the amino acid sequence or RYVRCRCI (SEQ ID NO: 70), or any combination thereof, and/or a modified peptide and/or fragment thereof; and/or combinations thereof, wherein each peptide present in the conjugate is covalently linked to at least one other peptide via a non-peptide linker, a peptide linker, and/or a cysteine-cysteine linkage.

14. The conjugate of claim 13, wherein the conjugate comprises a branched conjugate, a flanking conjugate, a single conjugate, a linear polymer, a bottlebrush polymer, or any combination thereof.

15. The conjugate of claim 13, wherein the conjugate is polymer-functionalized, encapsulated in a particle, embedded in and/or on a solid support, optionally wherein the peptide is formulated for release from the solid support, impregnated in a dressing, optionally wherein the peptide is formulated for release from the dressing, and/or is formulated for use in a nebulizer, for topical administration, and/or for systemic administration.

16. The conjugate of claim 9, wherein at least one peptide is a modified peptide that comprises a modification at the N-terminus, the C-terminus, or both the N-terminus and the C-terminus, optionally wherein the N-terminal and/or the C-terminal modification is selected from the group consisting of an addition or an aminohexanoic acid (AHX), an azido alanine (Ala(N₃)), a benzoylbenzoic acid (4-BBA), an azido phenylalanine (Phe(4-N₃)), a diazirine-containing amino acid, and a propargylglycine (PRA), and/or is a replacement of a phenylalanine with an azido phenylalanine (Phe(4-N₃)), further optionally wherein the peptide that is modified is selected from the group consisting of SEQ ID NOs: 19, 20, 40, 42, and 58.

17. A pharmaceutical composition comprising, consisting essentially of, or consisting of the peptide of claim 1 and a pharmaceutically acceptable carrier, diluent, or excipient.

18. The pharmaceutical composition of claim 17, wherein the pharmaceutical composition is pharmaceutically acceptable for use in a human.

19. A medical device comprising a support layer with an antibacterial agent embedded therein or associated therewith, wherein the antibacterial agent comprises the peptide of claim 1, optionally wherein the medical device is a wound dressing.

20. The medical device of claim 19, wherein the peptide, chimeric peptide, and/or conjugate is encapsulated in a particle that is embedded in or associated with the support layer.

21. A method for inhibiting the growth of and/or killing a bacterium, the method comprising contacting the bacterium with an effective amount of an antibacterial agent selected from the group consisting of the peptide of claim 1.

22. The method of claim 21, wherein the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to a Salmonella enterica serovars such as but not limited to Salmonella enterica serovar Typhi and Shigella flexneri; and biothreat agents such as but not limited to Bacillus anthracis in both vegetative and spore forms.

23. A method for treating or preventing a community and/or nosocomial infection in a subject, the method comprising administering to the subject a composition comprising a peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof.

24. A method for treating a bacterial infection present in a wound, the method comprising contacting the wound with an effective amount of a composition comprising one or more peptides, each peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof.

25. A method for treating a pulmonary infection in a subject, the method comprising administering to a subject in need thereof an effective amount of a composition comprising one or more peptides, each peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof.

26. The method of claim 25, wherein the composition is administered to the subject intranasally, by inhalation, optionally wherein the one or more peptides in the composition is/are aerosolized, or a combination thereof.

27. A method for treating or preventing a systemic bacterial infection in a subject, the method comprising administering to a subject in need thereof an effective amount of a composition comprising one or more peptides, each peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof.

28. The method of claim 21, further comprising administering to the subject a conventional antibiotic.

29. A method for inhibiting the growth of a biofilm, the method comprising contacting the biofilm with an effective amount of an antibacterial agent selected from the group consisting of the peptide of claim 1.

30. Use of the peptide of claim 1 for preventing or treating a bacterial infection.

31. A composition comprising, consisting essentially of, or consisting of a peptide comprising, consisting essentially of, or consisting of the amino acid sequence as set forth in any one of SEQ ID NOs: 2-151, optionally any one of SEQ ID NOs: 42, 45, 46, 48, 59, 70, 72, and 73, further optionally any one of SEQ ID NOs 48, 59, and 70, a modified peptide thereof, a fragment thereof, or any combination thereof, a conjugate thereof, a polymer thereof, or a combination thereof.

32. A pharmaceutical composition comprising, consisting essentially of, or consisting of the conjugate of claim 9 and a pharmaceutically acceptable carrier, diluent, or excipient.

33. A medical device comprising a support layer with an antibacterial agent embedded therein or associated therewith, wherein the antibacterial agent comprises the conjugate of claim 9, optionally wherein the medical device is a wound dressing.

34. A method for inhibiting the growth of and/or killing a bacterium, the method comprising contacting the bacterium with an effective amount of an antibacterial agent comprising the conjugate of claim 9.

35. The method of claim 23, further comprising administering to the subject a conventional antibiotic.

36. The method of claim 24, further comprising administering to the subject a conventional antibiotic.

37. The method of claim 25, further comprising administering to the subject a conventional antibiotic.

38. The method of claim 27, further comprising administering to the subject a conventional antibiotic.

39. A method for inhibiting the growth of a biofilm, the method comprising contacting the biofilm with an effective amount of an antibacterial agent selected from the group consisting of the conjugate of claim 9.

40. Use of the conjugate of claim 9 for preventing or treating a bacterial infection.


SEQUENCE LISTING (151 sequences)

SEQ ID NO: 1 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 2 - protein, 14 aa, synthetic construct
VPLSRTVRCTCISI

SEQ ID NO: 3 - protein, 15 aa, synthetic construct
TCISISNQPVNPRSL

SEQ ID NO: 4 - protein, 15 aa, synthetic construct
NPRSLEKLEIIPASQ

SEQ ID NO: 5 - protein, 15 aa, synthetic construct
IPASQFCPRVEIIAT

SEQ ID NO: 6 - protein, 15 aa, synthetic construct
EIIATMKKKGEKRCL

SEQ ID NO: 7 - protein, 15 aa, synthetic construct
EKRCLNPESKAIKNL

SEQ ID NO: 8 - protein, 16 aa, synthetic construct
KNLLKAVSKERSKRSP

SEQ ID NO: 9 - protein, 22 aa, synthetic construct
PESKAIKNLLKAVSKERSKRSP

SEQ ID NO: 10 - protein, 13 aa, synthetic construct
PLSRTVRCTCISI

SEQ ID NO: 11 - protein, 12 aa, synthetic construct
LSRTVRCTCISI

SEQ ID NO: 12 - protein, 11 aa, synthetic construct
SRTVRCTCISI

SEQ ID NO: 13 - protein, 10 aa, synthetic construct
RTVRCTCISI

SEQ ID NO: 14 - protein, 9 aa, synthetic construct
TVRCTCISI

SEQ ID NO: 15 - protein, 8 aa, synthetic construct
VRCTCISI

SEQ ID NO: 16 - protein, 7 aa, synthetic construct
RCTCISI

SEQ ID NO: 17 - protein, 6 aa, synthetic construct
CTCISI

SEQ ID NO: 18 - protein, 9 aa, synthetic construct
RTVRCTCIS

SEQ ID NO: 19 - protein, 8 aa, synthetic construct
RTVRCTCI

SEQ ID NO: 20 - protein, 7 aa, synthetic construct
RTVRCTC

SEQ ID NO: 21 - protein, 6 aa, synthetic construct
RTVRCT

SEQ ID NO: 22 - protein, 8 aa, synthetic construct
ATVRCTCI

SEQ ID NO: 23 - protein, 8 aa, synthetic construct
RAVRCTCI

SEQ ID NO: 24 - protein, 8 aa, synthetic construct
RTARCTCI

SEQ ID NO: 25 - protein, 8 aa, synthetic construct
RTVACTCI

SEQ ID NO: 26 - protein, 8 aa, synthetic construct
RTVRATCI

SEQ ID NO: 27 - protein, 8 aa, synthetic construct
RTVRCACI

SEQ ID NO: 28 - protein, 8 aa, synthetic construct
RTVRCTAI

SEQ ID NO: 29 - protein, 8 aa, synthetic construct
RTVRCTCA

SEQ ID NO: 30 - protein, 8 aa, synthetic construct
RRVRCTCI

SEQ ID NO: 31 - protein, 8 aa, synthetic construct
RNVRCTCI

SEQ ID NO: 32 - protein, 8 aa, synthetic construct
RDVRCTCI

SEQ ID NO: 33 - protein, 8 aa, synthetic construct
RCVRCTCI

SEQ ID NO: 34 - protein, 8 aa, synthetic construct
REVRCTCI

SEQ ID NO: 35 - protein, 8 aa, synthetic construct
RQVRCTCI

SEQ ID NO: 36 - protein, 8 aa, synthetic construct
RGVRCTCI

SEQ ID NO: 37 - protein, 8 aa, synthetic construct
RHVRCTCI

SEQ ID NO: 38 - protein, 8 aa, synthetic construct
RIVRCTCI

SEQ ID NO: 39 - protein, 8 aa, synthetic construct
RLVRCTCI

SEQ ID NO: 40 - protein, 8 aa, synthetic construct
RKVRCTCI

SEQ ID NO: 41 - protein, 8 aa, synthetic construct
RMVRCTCI

SEQ ID NO: 42 - protein, 8 aa, synthetic construct
RFVRCTCI

SEQ ID NO: 43 - protein, 8 aa, synthetic construct
RPVRCTCI

SEQ ID NO: 44 - protein, 8 aa, synthetic construct
RSVRCTCI

SEQ ID NO: 45 - protein, 8 aa, synthetic construct
RWVRCTCI

SEQ ID NO: 46 - protein, 8 aa, synthetic construct
RYVRCTCI

SEQ ID NO: 47 - protein, 8 aa, synthetic construct
RVVRCTCI

SEQ ID NO: 48 - protein, 8 aa, synthetic construct
RTVRCRCI

SEQ ID NO: 49 - protein, 8 aa, synthetic construct
RTVRCNCI

SEQ ID NO: 50 - protein, 8 aa, synthetic construct
RTVRCDCI

SEQ ID NO: 51 - protein, 8 aa, synthetic construct
RTVRCCCI

SEQ ID NO: 52 - protein, 8 aa, synthetic construct
RTVRCECI

SEQ ID NO: 53 - protein, 8 aa, synthetic construct
RTVRCQCI

SEQ ID NO: 54 - protein, 8 aa, synthetic construct
RTVRCGCI

SEQ ID NO: 55 - protein, 8 aa, synthetic construct
RTVRCHCI

SEQ ID NO: 56 - protein, 8 aa, synthetic construct
RTVRCICI

SEQ ID NO: 57 - protein, 8 aa, synthetic construct
RTVRCLCI

SEQ ID NO: 58 - protein, 8 aa, synthetic construct
RTVRCKCI

SEQ ID NO: 59 - protein, 8 aa, synthetic construct
RTVRCMCI

SEQ ID NO: 60 - protein, 8 aa, synthetic construct
RTVRCFCI

SEQ ID NO: 61 - protein, 8 aa, synthetic construct
RTVRCPCI

SEQ ID NO: 62 - protein, 8 aa, synthetic construct
RTVRCSCI

SEQ ID NO: 63 - protein, 8 aa, synthetic construct
RTVRCWCI

SEQ ID NO: 64 - protein, 8 aa, synthetic construct
RTVRCYCI

SEQ ID NO: 65 - protein, 8 aa, synthetic construct
RTVRCVCI

SEQ ID NO: 66 - protein, 8 aa, synthetic construct
RFVRCRCI

SEQ ID NO: 67 - protein, 8 aa, synthetic construct
RFVRCMCI

SEQ ID NO: 68 - protein, 8 aa, synthetic construct
RWVRCRCI

SEQ ID NO: 69 - protein, 8 aa, synthetic construct
RWVRCMCI

SEQ ID NO: 70 - protein, 8 aa, synthetic construct
RYVRCRCI

SEQ ID NO: 71 - protein, 8 aa, synthetic construct
RYVRCMCI

SEQ ID NO: 72 - protein, 8 aa, synthetic construct
RRVRCRCI

SEQ ID NO: 73 - protein, 8 aa, synthetic construct
RWVRCWCI

SEQ ID NO: 74 - protein, 9 aa, synthetic construct
  VARIANT 9: note = peptide is optionally biotinylated at the C-terminus
RTVRCTCIK

SEQ ID NO: 75 - protein, 9 aa, synthetic construct
FRTVRCTCI

SEQ ID NO: 76 - protein, 9 aa, synthetic construct
  VARIANT 1: note = amino acid 1 is optionally modified with a 4N3 moiety; note = peptide is optionally conjugated to a D-PRA moiety at the C-terminus
FRTVRCTCI

SEQ ID NO: 77 - protein, 9 aa, synthetic construct
  VARIANT 9: note = peptide is optionally modified at the C-terminus with a D-PRA moiety
  VARIANT 1: note = peptide is optionally modified at the N-terminus with a 4N3 moiety
FRRVRCTCI

SEQ ID NO: 78 - protein, 9 aa, synthetic construct
  VARIANT 9: note = peptide is optionally modified at the C-terminus with an N3 moiety
DRTVRCTCI

SEQ ID NO: 79 - protein, 9 aa, synthetic construct
  VARIANT 1: note = peptide is optionally modified at the N-terminus with a 4N3 moiety
  VARIANT 9: note = peptide is optionally modified at the C-terminus with an N3 moiety
FRTVRCTCI

SEQ ID NO: 80 - protein, 10 aa, synthetic construct
  VARIANT 1: note = peptide is optionally modified at the N-terminus with a 4N3 moiety
  VARIANT 10: note = peptide is optionally modified at the C-terminus with an N3 moiety
FRRVRCTCIA

SEQ ID NO: 81 - protein, 10 aa, synthetic construct
  VARIANT 1: note = peptide is optionally modified at the N-terminus with a 4N3 moiety
  VARIANT 10: note = peptide is optionally modified at the C-terminus with an N3 moiety
FRRVRCRCIA

SEQ ID NO: 82 - protein, 9 aa, synthetic construct
  VARIANT 1: note = peptide is optionally modified at the N-terminus with an N3 moiety
ARTVRCTCI

SEQ ID NO: 83 - protein, 10 aa, synthetic construct
  VARIANT 1: note = peptide is optionally modified at the N-terminus with an N3 moiety
  VARIANT 10: note = peptide is optionally modified at the C-terminus with a 4N3 moiety
ARTVRCTCIF

SEQ ID NO: 84 - protein, 10 aa, synthetic construct
  VARIANT 1: note = peptide is optionally modified at the N-terminus with an N3 moiety
  VARIANT 10: note = peptide is optionally modified at the N-terminus with a 4N3 moiety
ARRVRCTCIF

SEQ ID NO: 85 - protein, 10 aa, synthetic construct
  VARIANT 1: note = 1; note = peptide is optionally modified at the N-terminus with an N3 moiety
  VARIANT 10: note = peptide is optionally modified at the C-terminus with a 4N3 moiety
ARRVRCRCIF

SEQ ID NO: 86 - protein, 10 aa, synthetic construct
  VARIANT 1: note = peptide is optionally modified at the N-terminus with a photo-affinity moiety
  VARIANT 10: note = peptide is optionally modified at the C-terminus with an N3 moiety
MRTVRCTCIA

SEQ ID NO: 87 - protein, 10 aa, synthetic construct
  VARIANT 1: note = peptide is optionally modified at the N-terminus with a photo-affinity moiety
  VARIANT 10: note = peptide is optionally modified at the C-terminus with an N3 moiety
LRTVRCTCIA

SEQ ID NO: 88 - protein, 10 aa, synthetic construct
  VARIANT 1: note = peptide is optionally modified at the N-terminus with a photo-affinity moiety
  VARIANT 10: note = peptide is optionally modified at the C-terminus with an N3 moiety
KRTVRCTCIA

SEQ ID NO: 89 - protein, 10 aa, synthetic construct
  VARIANT 1: note = peptide is optionally modified at the N-terminus with an N3 moiety
  VARIANT 10: note = peptide is optionally modified at the C-terminus with a photo-affinity moiety
ARTVRCTCIM

SEQ ID NO: 90 - protein, 10 aa, synthetic construct
  VARIANT 1: note = peptide is optionally modified at the N-terminus with an N3 moiety
  VARIANT 10
ARTVRCTCIL

SEQ ID NO: 91 - protein, 10 aa, synthetic construct
  VARIANT 1: note = peptide is optionally modified at the N-terminus with an N3 moiety
  VARIANT 10: note = peptide is optionally modified at the C-terminus with a photo-affinity moiety
ARTVRCTCIK

SEQ ID NO: 92 - protein, 9 aa, synthetic construct
  VARIANT 1: note = peptide is optionally modified at the N-terminus with a photo-affinity moiety
  VARIANT 9: note = peptide is optionally modified at the N-terminus with a D-PRA moiety
MRTVRCTCI

SEQ ID NO: 93 - protein, 9 aa, synthetic construct
  VARIANT 1: note = peptide is optionally modified at the N-terminus with a photo-affinity moiety
  VARIANT 9: note = peptide is optionally modified at the C-terminus with a D-PRA moiety
LRTVRCTCI

SEQ ID NO: 94 - protein, 9 aa, synthetic construct
  VARIANT 1: note = peptide is optionally modified at the N-terminus with a photo-affinity moiety
  VARIANT 9: note = peptide is optionally modified at the C-terminus with a D-PRA moiety
KRTVRCTCI

SEQ ID NO: 95 - protein, 9 aa, synthetic construct
  VARIANT 1: note = peptide is optionally modified at the N-terminus with a D-PRA moiety
  VARIANT 9: note = peptide is optionally modified at the C-terminus with a photo-affinity moiety
RTVRCTCIM

SEQ ID NO: 96 - protein, 9 aa, synthetic construct
  VARIANT 1: note = peptide is optionally modified at the N-terminus with a D-PRA moiety
  VARIANT 9: note = peptide is optionally modified at the C-terminus with a photo-affinity moiety
RTVRCTCIL

SEQ ID NO: 97 - protein, 9 aa, synthetic construct
  VARIANT 1: note = peptide is optionally modified at the N-terminus with a D-PRA moiety
  VARIANT 9: note = peptide is optionally modified at the C-terminus with a photo-affinity moiety
RTVRCTCIK

SEQ ID NO: 98 - protein, 7 aa, synthetic construct
  VARIANT 2: note = amino acid 2 is optionally an allothreonine
TVRCTCI

SEQ ID NO: 99 - protein, 8 aa, synthetic construct
  VARIANT 2: note = amino acid 2 is optionally a methyl threonine
RTVRCTCI

SEQ ID NO: 100 - protein, 8 aa, synthetic construct
  VARIANT 2: note = the X as position 2 has an aminobutyric acid moiety
RXVRCTCI

SEQ ID NO: 101 - protein, 8 aa, synthetic construct
  VARIANT 2: note = the alanine at position 2 is optionally a napthylalanine
RAVRCTCI

SEQ ID NO: 102 - protein, 8 aa, synthetic construct
  VARIANT 6: note = the threonine at position 6 is optionally an allothreonine
RTVRCTCI

SEQ ID NO: 103 - protein, 8 aa, synthetic construct
  VARIANT 6: note = the threonine at position 6 is optionally a methyl threonine
RTVRCTCI

SEQ ID NO: 104 - protein, 8 aa, synthetic construct
  VARIANT 6: note = the X at position 6 is optionally an aminobutyric acid moiety
RTVRCXCI

SEQ ID NO: 105 - protein, 8 aa, synthetic construct
  VARIANT 6: note = the alanine at position 6 is optionally a napthyl alanine
RTVRCACI

SEQ ID NO: 106 - protein, 8 aa, synthetic construct
AYVRCRCI

SEQ ID NO: 107 - protein, 8 aa, synthetic construct
NYVRCRCI

SEQ ID NO: 108 - protein, 8 aa, synthetic construct
DYVRCRCI

SEQ ID NO: 109 - protein, 8 aa, synthetic construct
CYVRCRCI

SEQ ID NO: 110 - protein, 8 aa, synthetic construct
QYVRCRCI

SEQ ID NO: 111 - protein, 8 aa, synthetic construct
EYVRCRCI

SEQ ID NO: 112 - protein, 8 aa, synthetic construct
GYVRCRCI

SEQ ID NO: 113 - protein, 8 aa, synthetic construct
HYVRCRCI

SEQ ID NO: 114 - protein, 8 aa, synthetic construct
IYVRCRCI

SEQ ID NO: 115 - protein, 8 aa, synthetic construct
LYVRCRCI

SEQ ID NO: 116 - protein, 8 aa, synthetic construct
KYVRCRCI

SEQ ID NO: 117 - protein, 8 aa, synthetic construct
MYVRCRCI

SEQ ID NO: 118 - protein, 8 aa, synthetic construct
FYVRCRCI

SEQ ID NO: 119 - protein, 8 aa, synthetic construct
PYVRCRCI

SEQ ID NO: 120 - protein, 8 aa, synthetic construct
SYVRCRCI

SEQ ID NO: 121 - protein, 8 aa, synthetic construct
TYVRCRCI

SEQ ID NO: 122 - protein, 8 aa, synthetic construct
WYVRCRCI

SEQ ID NO: 123 - protein, 8 aa, synthetic construct
YYVRCRCI

SEQ ID NO: 124 - protein, 8 aa, synthetic construct
VYVRCRCI

SEQ ID NO: 125 - protein, 7 aa, synthetic construct
  VARIANT 1: note = peptide is optionally modified at the N-terminus with a citrulline
YVRCRCI

SEQ ID NO: 126 - protein, 8 aa, synthetic construct
  VARIANT 1: note = the arginine at position 1 is optionally homoarginine
RYVRCRCI

SEQ ID NO: 127 - protein, 8 aa, synthetic construct
  VARIANT 1: note = the arginine at position 1 is optionally nitroarginine
RYVRCRCI

SEQ ID NO: 128 - protein, 8 aa, synthetic construct
  VARIANT 1: note = the X at position 1 is optionally norarginine
XYVRCRCI

SEQ ID NO: 129 - protein, 8 aa, synthetic construct
RAVRCRCI

SEQ ID NO: 130 - protein, 8 aa, synthetic construct
RRVRCRCI

SEQ ID NO: 131 - protein, 8 aa, synthetic construct
RNVRCRCI

SEQ ID NO: 132 - protein, 8 aa, synthetic construct
RDVRCRCI

SEQ ID NO: 133 - protein, 8 aa, synthetic construct
RCVRCRCI

SEQ ID NO: 134 - protein, 8 aa, synthetic construct
RQVRCRCI

SEQ ID NO: 135 - protein, 8 aa, synthetic construct
REVRCRCI

SEQ ID NO: 136 - protein, 8 aa, synthetic construct
RGVRCRCI

SEQ ID NO: 137 - protein, 8 aa, synthetic construct
RHVRCRCI

SEQ ID NO: 138 - protein, 8 aa, synthetic construct
RIVRCRCI

SEQ ID NO: 139 - protein, 8 aa, synthetic construct
RLVRCRCI

SEQ ID NO: 140 - protein, 8 aa, synthetic construct
RKVRCRCI

SEQ ID NO: 141 - protein, 8 aa, synthetic construct
RMVRCRCI

SEQ ID NO: 142 - protein, 8 aa, synthetic construct
RFVRCRCI

SEQ ID NO: 143 - protein, 8 aa, synthetic construct
RPVRCRCI

SEQ ID NO: 144 - protein, 8 aa, synthetic construct
RSVRCRCI

SEQ ID NO: 145 - protein, 8 aa, synthetic construct
RTVRCRCI

SEQ ID NO: 146 - protein, 8 aa, synthetic construct
RWVRCRCI

SEQ ID NO: 147 - protein, 8 aa, synthetic construct
RVVRCRCI

SEQ ID NO: 148 - protein, 8 aa, synthetic construct
  VARIANT 2: note = the tyrosine at position 2 is optionally methyl tyrosine
RYVRCRCI

SEQ ID NO: 149 - protein, 8 aa, synthetic construct
  VARIANT 2: note = the tyrosine at position 2 is optionally iodotyrosine
RYVRCRCI

SEQ ID NO: 150 - protein, 8 aa, synthetic construct
  VARIANT 2: note = the tyrosine at position 2 is optionally dimethyl tyrosine
RYVRCRCI

SEQ ID NO: 151 - protein, 8 aa, synthetic construct
  VARIANT 2: note = the proline at position 2 is optionally homoproline
RPVRCRCI